(19) United States
(12) Patent Application Publication
(10) Pub. No.: US 2010/0063262 A1
(43) Pub. Date: Mar. 11, 2010

(54) PROCESS FOR PRODUCING 5-AMINOPYRAZOLE DERIVATIVE, AND AZO DYE

(75) Inventors: Keiichi Tateishi, Minami-Ashigara-shi (JP); Masaru Takasaki, Minami-Ashigara-shi (JP); Shigeaki Tanaka, Minami-Ashigara-shi (JP); Tadao Sekioka, Minami-Ashigara-shi (JP); Takahiro Morita, Odawara-shi (JP)

Correspondence Address: SUGHRUE-265550, 2100 PENNSYLVANIA AVE. NW, WASHINGTON, DC, 20037-3213, US

(73) Assignee: FUJIFILM CORPORATION, Tokyo (JP)

(21) Appl. No.: 12/514,119

(22) PCT Filed: Nov. 9, 2007
(86) PCT No.: PCT/JP2007/072240

§ 371 Date: May 8, 2009

(30) Foreign Application Priority Data
Nov. 10, 2006 (JP) 2006-304998
Nov. 10, 2006 (JP) 2006-304999
Aug. 15, 2007 (JP) 2007-211816

(51) Int. Cl. C09B 27/00; C07D 231/14

(52) U.S. Cl. 534/586; 548/371.7

(57) ABSTRACT

A process for producing a 5-aminopyrazole derivative represented by Formula (I) includes a step of reacting a hydrazine compound represented by Formula (II) with a compound represented by Formula (III) under a condition satisfying Reaction Condition 1 and Reaction Condition 2, wherein
Reaction Condition 1 is that the reaction is conducted in a reaction solvent containing water, and
Reaction Condition 2 is that pH of a liquid mixture containing the hydrazine compound represented by Formula (II), the compound represented by Formula (III), the reaction solvent, and an acidifying agent upon charging is within a range of from 2.0 to 4.0 at 25° C.:

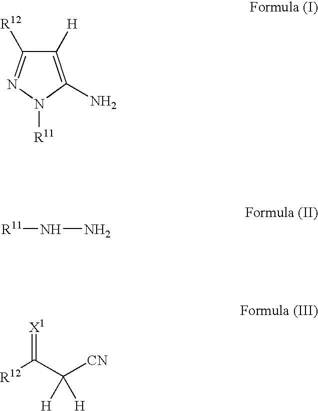

wherein the symbols in the formulae (I) to (III) are defined in the specification.

### TECHNICAL FIELD

The present invention relates to a process for producing a 5-aminopyrazole derivative substituted with a water-soluble group. More specifically, the invention relates to a process for producing a 5-aminopyrazole derivative (coupler component) substituted with a hydroxyl group, carboxyl group, or sulfo group in an inexpensive water solvent at a high yield and at a high purity and relates to a process for producing a 5-amino-4-cyanopyrazole derivative (diazo component) substituted with a hydroxyl group, carboxyl group, or sulfo group simply and conveniently at a high yield and a high purity.

Further, invention relates to a method of using a 5-aminopyrazole derivative. More specifically, the invention relates to a method of using the derivative for producing an azo dye of preferred hue and color by using the 5-aminopyrazole derivative.

### BACKGROUND ART

5-aminopyrazole derivatives are useful compounds as intermediate products for functional compounds such as photographic additives, sensitizing dyes, dyes, pigments, electronic materials, and medical and agricultural chemicals, and synthesis methods have been known long since (Gazz. chim. ital. 81, 380 (1951), Gazz. chim. ital. 82, 373 (1952), and “Chemistry for Heterocyclic Compound” (published on 1988-04-01) written by Hiroshi Yamanaka, et al.). Among the known synthesis methods, reaction of hydrazine compounds and a 1,3-dicarbonyl compounds such as β-ketoester or β-ketonitrile can be mentioned as typical and general purpose synthesis methods.

On the other hand, also for the synthesis method of 5-aminopyrazole derivatives (coupler component) substituted with water-soluble groups, they can be obtained by reaction between hydrazine compounds and β-ketonitrile. WO 06/082669 describes that they are reacted under a strongly acidic condition by using a mixed solvent of ethylene glycol and methanol. However, in a case of reaction under the strongly acidic condition, there is a problem that the purity is lowered since ethylene glycol is contained in crude crystals of obtained 5-aminopyrazole derivatives. Further, JP-A-2006-57076 describes reaction under an alkaline condition by using an aqueous solvent. However, the method involves a problem that the yield is extremely low since a great amount of by-products are also formed.

Since the hydrazine compound represented by Formula (II) is easily soluble to water in strongly acidic condition or alkaline condition, the reaction of the invention is usually conducted generally in a strongly acidic condition or neutral condition to alkaline condition. However, this involves a problem that the reaction per se does not proceed under the strongly acidic condition or the alkaline condition or by products are formed in a great amount depending on the type of the reaction system or the molecular structure of a reaction substrate and both the yield and the purity were not satisfactory. As described above, it has been found that the purity is low or the yield is low under the strongly acidic condition or the alkaline condition in a case of reaction with an aqueous solvent system which is inexpensive and gives less burden on the environment as a synthesis method of water-soluble group-substituted 5-amino pyrazole derivatives (coupler component).

Further, a synthesis method, particularly, for the 5-amino-4-cyano pyrazole derivative (diazo component) includes reaction between a hydrazine compound and an alkoxymethylene malononitrile as a typical synthesis method (described in U.S. Pat. No. 3,336,285). Further, JP-A-2006-057076 or WO 06/082669 describes examples of synthesis for obtaining 5-amino-4-cyanopyrazole derivatives substituted with carboxyl group using anilines substituted with a carboxyl group such as 5-aminoisophthalic acid as the starting material. However, this cannot always be said to be a production process which is simple and convenient and providing high yield and high purity since the yield is lowered due to the isolation loss and impurities are intruded inevitably because of extremely poor filterability to lower the purity in the step of isolation a hydrazine compound as an intermediate synthesis product. Further, since reaction of the obtained hydrazine compound and ethoxymethylene malononitrile is conducted in an organic solvent, this is a production process that gives a large burden on the environment with in view of liquid wastes, etc. As described above, in the process for producing 5-aminopyrazole derivative (diazo component) substituted with a water-soluble group, when the hydrazine compound substituted with water-soluble group formed as the intermediate synthesis product is isolated, the yield is lowered due to the isolation loss and the purity is lowered due to the worsening of the filterability and, further, the procedures are complicated to result in a problem of increasing the burden on the production.

### DISCLOSURE OF THE INVENTION

Accordingly, the present invention intends to provide a process for producing 5-aminopyrazole derivative (coupler component) substituted with a water-soluble group represented by Formula (I) at a high yield and at a high purity in an aqueous system which is inexpensive and gentle to the environment.

Further, the invention intends to provide a process for producing a 5-aminopyrazole derivative (diazo component) substituted with a water-soluble group represented by Formula (4) simply and conveniently and at a high yield and a high purity. Further, it intends to provide a method of using a 5-aminopyrazole derivative for producing an azo dye excellent in the hue and the color. Particularly, it intends to provide a method of using a 5-aminopyrazole derivative for producing a dye belonging to a specified range with respect to the absorption maximum and the half-value with of an absorption band in a visible absorption spectrum which are important for the hue and the color.

As a result of a study for overcoming such problems in the related art, it has been found that using water which is inexpensive and gentle to the environment as a reaction solvent and starting the reaction at a pH upon charging within a range of from 2.0 to 4.0 (25° C.) enable a 5-aminopyrazole derivative substituted with a water-soluble group represented by Formula (I) to be synthesized at a reduced cost and at a high yield and a high purity from the starting materials represented by Formula (II) and Formula (III).

Further, it has been found that the 5-aminopyrazole derivative substituted with a water-soluble group of Formula (4) can be synthesized at a high yield and a high purity even when a hydrazine compound represented by Formula (2) having extremely poor filterability is reacted as it is without isolation with a compound represented by Formula (3). Further, it has been found that when the 5-aminopyrazole derivative is used as an intermediate product for an azo dye, the hue and the color can be improved. That is, the foregoing objects of the invention have been attained by the following methods:

1. A process for producing a 5-aminopyrazole derivative represented by Formula (I), including:

a step of reacting a hydrazine compound represented by Formula (II) with a compound represented by Formula (III) under a condition satisfying Reaction Condition 1 and Reaction Condition 2, wherein

Reaction Condition 1 is that the reaction is conducted in a reaction solvent containing water, and

Reaction Condition 2 is that pH of a liquid mixture containing the hydrazine compound represented by Formula (II), the compound represented by Formula (III), the reaction solvent, and an acidifying agent upon charging is within a range of from 2.0 to 4.0 at 25° C.:

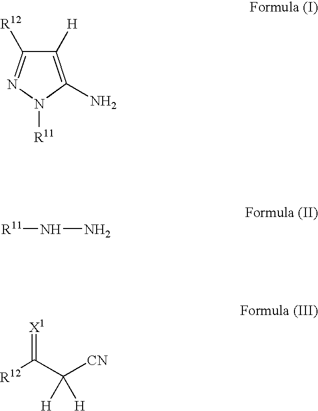

wherein

R¹¹ represents an aromatic group or a heterocyclic group, each of which is substituted with a hydroxyl group, a carboxyl group, or a sulfo group,

R¹² represents an aliphatic group, aromatic group, or heterocyclic group each of which is substituted or unsubstituted, and

X¹ represents an oxygen atom or NH.

“Upon charging” is defined herein as an instance the hydrazine compound represented by Formula (II), the compound represented by Formula (III), the reaction solvent, and the acidifying agent are mixed and stirred at 25° C. for 5 min, that is, an instance the total reaction components are mixed and stirred at 25° C. for 5 min.

2. The production process as described in above 1, wherein

R¹¹ in Formula (I) and Formula (II) is a heterocyclic group substituted with a hydroxyl group.

3. The production process as descried in above 1 or 2, wherein

the acidifying agent is sulfuric acid or hydrochloric acid.

4. The production process as described in any of above 1 to 3, wherein

the reaction is conducted at a temperature of from 30 to 70° C.

5. The production process according to any of above 1 to 4, wherein

the reaction solvent containing water is a solvent consisting only of water.

6. The production process according to any of above 1 to 5, wherein

pH of the liquid mixture upon charging is within a range of from 2.5 to 3.5 at 25° C.

7. A process for producing a 5-aminopyrazole derivative represented by Formula (4) including:

(A) a step of diazotizing an amine compound represented by Formula (1) to prepare a diazonium salt;

(B) a step of reducing the diazonium salt to prepare a hydrazine compound represented by Formula (2); and

(C) a step of reacting the hydrazine compound with a compound represented by Formula (3) to prepare a 5-aminopyrazole derivative represented by Formula (4),

wherein the steps (A) to (C) are conducted continuously:

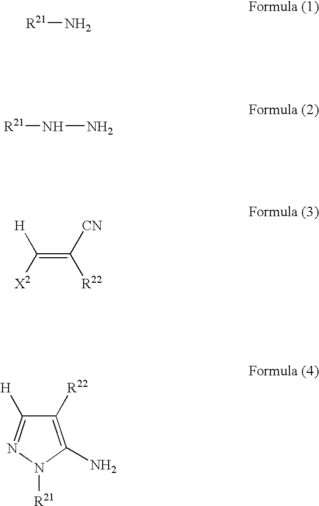

wherein

R²¹ represents an aromatic group or a heterocyclic group, each of which is substituted with a hydroxyl group, carboxyl group or sulfo group,

R²² represents an electron-withdrawing group, and

X² represents a halogen atom, hydroxyl group, amino group, or alkoxy group.

8. The production process as described in above 7, wherein

the steps (A) and (B) are conducted with a solvent consisting only of water.

9. The production process as described in above 7 or 8, wherein

all the steps (A), (B) and (C) are conducted with a solvent consisting only of water.

10. The production process as described in any of above 7 to 9, wherein

the reducing agent for reducing the diazonium salt in the step (B) is sodium sulfite or sodium disulfite.

11. The production process as described in any of above 7 to 10, wherein

the reducing agent for reducing the diazonium salt in the step (B) is added in an amount of from 1.2 to 3.0 mol based on 1 mol of the amine compound represented by Formula (1).

12. The production process as described in any of above 7 to 11, wherein

the step (C) is conducted in a reaction solution, pH of which is within a range of from 5.0 to 9.0.

13. The production process as described in any of above 7 to 12, further including after the step(C):

a step of crystallizing the 5-aminopyrazole derivative represented by Formula (4) prepared in the step (C) at pH of the reaction solution within a range of from 0.5 to 2.0, and

a step of collecting the crystallized 5-aminopyrazole derivative by filtration.

14. A production process as described in any one of above 7 to 13, wherein

a reaction mixture obtained by reacting a compound represented by Formula (6) with a compound represented by a Formula (7) is used instead of the compound represented by Formula (3) in the step (C):

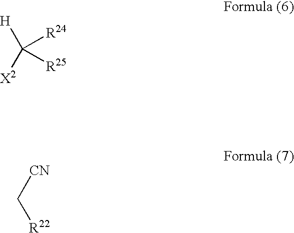

wherein

R²² represents an electron-withdrawing group,

X² represents a halogen atom, a hydroxyl group, amino group, or alkoxy group, and

R²⁴ and R²⁵ each represents independently a group leaving easily by the reaction of the compounds.

15. The production process as described in above 14, wherein

the step (C) is conducted in a reaction solution, pH of which is within a range of from 3.0 to 9.

16. A production process as described in above 14 or 15, further including after the step (C):

a step of crystallizing the 5-aminopyrazole derivative represented by Formula (4) in the step (C) at pH of the reaction solution within a range of from 3.0 to 5.0 and

a step of collecting the crystallized 5-aminopyrazole derivative by filtration.

17. An azo dye produced from the 5-aminopyrazole derivative produced by the production process as described in any of above 1 to 16.

18. A process for producing an azo dye, including:

a step of producing the 5-aminopyrazole derivative represented by Formula (I) by the production method according to any of claims 1 to 6;

a step of producing the 5-aminopyrazole derivative represented by Formula (4) by the production method according to any of claims 7 to 16; and

a step of reacting the 5-aminopyrazole derivative represented by Formula (I) with the 5-aminopyrazole derivative represented by Formula (4).

### BEST MODE FOR CARRYING OUT THE INVENTION

Preferred embodiment of the invention is to be described specifically.

At first, the substituent in the present specification is to be described briefly. The substituent in the present specification includes the following groups (the groups are referred to as “substituent A”).

For example, they include at halogen atom, alkyl group, alkenyl group, alkynyl group, allyl group, heterocyclic group, cyano group, hydroxyl group, nitro group, carboxyl group, alkoxy group, aryloxy group, sillyloxy group, heterocyclooxy group, acyloxy group, carbamoyloxy group, alkoxycarbonyloxy group, aryloxycarbonyloxy group, amino group, acylamino group, aminocarbonyl amino group, alkoxycarbonyl amino group, aryloxycarbonyl amino group, sulfamoyl amino group, alkyl or aryl sulfonyl amino group, mercapto group, alkylthio group, arylthio group, heterocyclic thio group, sulfamoyl group, sulfo group, alkyl or aryl sulfinyl group, alkyl or aryl sulfonyl group, acyl group, aryloxycarbonyl group, alkoxycarbonyl group, carbamoyl group, aryl or heterocyclic azo group, imido group, phosphino group, phosphinyl group, phosphinyloxy group, phosphinyl amino group, and silyl group.

The aliphatic group used in the present specification means an alkyl group, substituted alkyl group, alkenyl group, substituted alkenyl group, alkynyl group, and substituted alkynyl group. Further, the aromatic group used in the present specification means aryl group, and substituted aryl group.

More specifically, the halogen atom includes, for example, a fluorine atom, chlorine atom, bromine atom, and iodine atom.

The alkyl group includes linear, branched, or cyclic substituted or non-substituted alkyl groups and also includes cycloalkyl group, bicycloalkyl group, and, tricycle structure with more cyclic structure. The alkyl group in the substituent to be described later (for example, alkyl group in the alkoxy group or alkyl thio group) also represents the alkyl group of such meaning. Specifically, the alkyl group includes, preferably, alkyl groups of from 1 to 30 carbon atoms, for example, methyl group, ethyl group, n-propyl group, isopropyl group, t-butyl group, n-octyl group, eicosyl group, 2-chloroethyl group, 2-cyanoethyl group, and 2-ethylhexyl group, the cycloalkyl group includes, preferably, substituted or non-substituted cycloalkyl groups of 3 to 30 carbon atoms, for example, cyclohexyl group, cyclopentyl group, and 4-n-dodecylcycloalkyl group. The bicycloalkyl group includes, preferably, substituted or non-substituted bicycloalkyl groups having 5 to 30 carbon atoms, that is, this includes, for example, a monovalent group formed by removing one hydrogen atom from a bicycloalkane having 5 to 30 carbon atoms, for example, bicycle[1,2,2]heptane-2-yl group, bicycle[2,2,2]octane-3-yl group, etc.

The alkenyl group includes linear, branched or cyclic substituted or non-substituted alkenyl groups and include a cycloalkenyl group and bicycloalkenyl group. Specifically, the alkenyl group includes, preferably, substituted or non-substituted alkenyl groups of from 2 to 30 carbon atoms, for example, a vinyl group, aryl group, prenyl group, geranyl group, and oleyl group, the cycloalkenyl group includes, preferably, substituted or non-substituted cycloalkenyl groups of from 3 to 30 carbon atoms, that is, a monovalent group formed by removing one hydrogen atom from a cycloalkene having from 3 to 30 carbon atoms, for example, 2-cyclopentene-1-yl group, and 2-cyclohexene-1-yl group, and the bicycloalkenyl group includes substituted or non-substituted bicycloalkenyl groups, preferably, substituted or non-substituted bicyloalkenyl groups of from 5 to 30 carbon atoms, that is, a monovalent group formed by removing one hydrogen atom from a bicycloalkene having one double bond, for example, bicycle[2,2,1]hepto-2-en-1-yl group, bicycle[2,2,2]octo-2-en-4-yl group, etc.

The alkynyl group includes, preferably, substituted or non-substituted alkynyl groups of 2 to 30 carbon atoms, for example, ethynyl group, propargyl group, and trimethylsilylethynyl group.

The aryl group includes, preferably, substituted or non-substituted aryl groups of 6 to 30 carbon atoms, for example, phenyl group, p-tolyl group, naphthyl group, m-chlorophenyl group, and o-hexadenoyl aminophenyl group.

The heterocyclic group is, preferably, a monovalent group formed by removing one hydrogen atom from a 5- or 6-membered substituted or non-substituted aromatic or non-aromatic heterocyclic compound and, more preferably, a 5- or 6-membered heterocyclic group of 3 to 30 carbon atoms and may be either a single nuclear structure or a polynuclear structure in which two or more rings are condensated. Further, as the heterocyclic group, heterocyclic groups containing at least one of N, O, S atoms are preferred. They include, for example, thienyl group, furyl group, pyrrolyl group, indolyl group, imidazolyl group, benzimidazolyl group, pyrazolyl group, indozolyl group, thiazolyl group, benzothiazolyl group, isothiazolyl group, benzoisothiazolyl group, oxazolyl group, benzoxazolyl group, isooxazolyl group, 1,2,4-thiadiazolyl group, 1,3,4-thiadiazolyl group, 1,2,4-oxadiazolyl group, 1,3,4-oxadiazolyl group, triazolyl group, piridyl group, pirazyl group, pirimidyl group, piridazyl group, 1,3,5-triazyl group, quinolyl group, isoquinolyl group, and phthaladinyl group.

The alkoxy group includes, preferably, substituted or non-substituted alkoxy groups of 1 to 30 carbon atoms, for example, methoxy group, ethoxy group, isopropoxy group, t-butoxy group, n-octyloxy group, and 2-methoxyethoxyl group.

The aryloxy group includes, for example, substituted or non-substituted aryloxy groups of 6 to 30 carbon atoms, for example, phenoxy group, 2-methylphenoxy group, 4-t-butylphenoxy group, 3-nitrophenoxy group, and 2-tetradecanoylaminophenyl group.

The silyloxy group includes, for example, substituted or non-substituted silyloxy groups of 0 to 20 carbon atoms, for example, trimethylsilyloxy group and diphenylmethylsilyloxy group.

The heterocyclicoxy group includes, preferably, substituted or non-substituted heterocyclic oxy groups of 2 to 30 carbon atoms, for example, 1-phenyltetrazole-5-oxy group and 2-tetrahydropyranyloxy group.

Acyloxy group includes, preferably, substituted or non-substituted alkylcarbonyloxy groups of 2 to 30 carbon atoms, substituted or non-substituted aryl carbonyloxy group of 6 to 30 carbon atoms, for example, acetyloxy group, pivaloyl group, stearoyloxy group, benzoyloxy group, and p-methoxyphenyl carbonyloxy group.

The carbamoyloxy group includes, preferably, substituted or non-substituted carbamoyloxy groups of 1 to 30 carbon atoms, for example, N,N-dimethylcarbamoyloxy group, N,N-diethylcarbamoyloxy group, morpholinocarbonyloxy group, N,N-di-n-octylaminocarbonyloxy group, and N-n-octylcarbamoyloxy group.

The alkoxycarbonyloxy group includes, preferably, substituted or non-substituted alkoxycarbonyloxy groups of 2 to 30 carbon atoms, for example, methoxycarbonyloxy group, ethoxycarbonyloxy group, t-butoxycarbonyloxy group, and n-octylcarbonyloxy group.

The aryloxycarbonyloxy group includes, preferably, substituted or non-substituted aryloxy carbonyloxy groups of 7 to 30 carbon atoms, for example, phenoxycarbonyloxy group, p-methoxyphenoxy carbonyloxy group, and p-n-hexadecyloxyphenoxy carbonyloxy group.

The amino group includes alkylamino groups, arylamino groups, heterocyclic amino groups and includes, preferably, amino group, substituted or non-substituted alkylamino groups of 1 to 30 carbon atoms, substituted or non-substituted anilino groups of 6 to 30 carbon atoms, for example, methylamino group, dimethylamino group, aniline group, N-methyl-anilino group, and diphenylamino group.

The acylamino group includes, preferably, formyl amino group, substituted or non-substituted alkylcarbonyl amino groups of 1 to 30 carbon atoms and substituted or non-substituted arylcarbonyl amino groups of 6 to 30 carbon atoms, for example, acetylamino group, pivaloylamino group, lauroylamino group, benzoylamino group, and 3,4,5-tri-n-octyloxyphenyl carbonylamino group.

The aminocarbonylamino group includes, for example, substituted or non-substituted aminocarbonyl amino groups of 1 to 30 carbon atoms, for example, carbamoyl amino group, N,N-dimethylamino carbonylamino group, N,N-diethylamino carbonylamino group, and morpholino carbonylamino group.

The alkoxycarbonyl amino group includes, preferably, substituted or non-substituted alkoxycarbonylamino groups of 2 to 30 carbon atoms, for example, methoxycarbonylamino group, ethoxycarbonylamino group, t-butoxycarbonylamino group, n-octadecyloxy carbonylamino group, and N-methyl-methoxy carbonylamino group.

The aryloxy carbonylamino group includes, preferably, substituted or non-substituted aryloxy carbonylamino groups of 7 to 30 carbon atoms, for example, phenoxycarbonylamino group, p-chlorophenoxycarbonylamino group, and m-n-octyloxyphenoxy carbonylamino group.

The sulfamoylamino group includes, preferably, substituted or non-substituted sulfamonylamino groups of 0 to 30 carbon atoms, for example, sulfamolyamino group, N,N-dimethylamino sulfonylamino group, and N-n-octylamino sulfonylamino group.

The alkyl or aryl sulfonylamino group includes, preferably, substituted or non-substituted alkylsulfonylamino groups of 1 to 30 carbon atoms and substituted or non-substituted arylsulfonylamino groups of 6 to 30 carbon atom, for example, methylsulfonylamino group, butylsulfonylamino group, phenylsulfonylamino group, 2,3,5-trichlorophenyl sulfonylamino group, and p-methylphenyl sulfonylamino group.

The alkylthio group includes, preferably, substituted or non-substituted alkylthio groups of 1 to 30 carbon atoms, for example, methylthio group, ethylthio group, and n-hexadecylthio group.

The arylthio group includes, preferably, substituted or non-substituted arylthio groups of 6 to 30 carbon atoms, for example, phenylthio group, p-chlorophenylthio group, and m-methoxyphenylthio group.

The heterocyclic thio group includes, preferably, substituted or non-substituted heterocyclic thio groups of 2 to 30 carbon atoms, for example, 2-benzothizolyl group, and 1-phenyltetrazole-5-yl thio group.

The sulfamoyl group includes, for example, substituted or non-substituted sulfamoyl groups of 0 to 30 carbon atoms, for example, N-ethylsulfamoyl group, N-(3-dodecyloxypropyl)sulfamoyl group, N,N-dimethylsulfamoyl group, N-acetylsulfamoyl group, N-benzoylsulfamoyl group, and N—(N'-phenylcarbamoyl)sulfamoyl group.

The alkyl or arylsulfinyl group includes, preferably, substituted or non-substituted alkylsulfinyl groups of 1 to 30 carbon atoms and substituted or non-substituted arylsulfinyl groups of 6 to 30 carbon atoms, for example, methylsulfinyl group, ethylsulfinyl group, phenylsulfinyl group, and p-methylphenylsulfinyl group.

The alkyl or arylsulfonyl group includes, preferably, substituted or non-substituted alkylsulfonyl groups of 1 to 30 carbon atoms and substituted or non-substituted arylsulfonyl groups of 6 to 30 carbon atoms, for example, methylsulfonyl group, ethylsulfonyl group, phenylsulfonyl group, and p-methylphenylsulfonyl group.

The acyl group includes, preferably, formyl group, substituted or non-substituted alkylcarbonyl groups of 2 to 30 carbon atoms, substituted or non-substituted arylcarbonyl groups of 7 to 30 carbon atoms, substituted or non-substituted heterocyclic carbonyl groups of 2 to 30 carbon atoms bonded at the carbon atom with a carbonyl group, for example, acetyl group, pivaloyl group, 2-chloroacetyl group, stearolyl group, benzoyl group, p-n-octyloxyphenyl carbonyl group, 2-pyridylcarbonyl group, and 2-furylcarbonyl group.

The aryloxycarbonyl group includes, preferably, substituted or non-substituted aryloxycarbonyl groups of 7 to 30 carbon atoms, for example, phenoxycarbonyl group, o-chlorophenoxycarbonyl group, m-nitrophenoxycarbonyl group, and p-t-butylphenoxycarbonyl group.

The alkoxycarbonyl group includes, preferably, substituted or non-substituted alkoxycarbonyl groups of 2 to 30 carbon atom, for example, methoxycarbonyl group, ethoxycarbonyl group, t-butoxycarbonyl group, and n-octadecyloxycarbonyl group.

The carbamoyl group includes, preferably, substituted or non-substituted carbamoyl groups of 1 to 30 carbon atoms, for example, carbamoyl group, N-methylcarbamoyl group, N,N-dimethylcarbamoyl group, N,N-di-n-octylcarbamoyl group, and N-(methylsulfonyl)carbamoyl group.

The aryl or heterocyclic azo group includes, preferably, substituted or non-substituted arylazo groups of 6 to 30 carbon atoms or substituted or non-substituted heterocyclic azo groups of 3 to 30 carbon atoms, for example, phenylazo, p-chlorophenyl azo, and 5-ethylthio-1,3,4-thiadiazole-2-yl azo.

The imido group includes, preferably, N-succinimide group, N-phthalimide group, etc.

The phosphino group includes, preferably, substituted or non-substituted phosphino groups of 0 to 30 carbon atoms, for example, dimethylphosphino group, diphenylphosphino group, and methylphenoxyphosphino group.

The phosphinyl group includes, preferably, substituted or non-substituted phosphinyl groups of 0 to 30 carbon atoms, for example, phosphinyl group, dioctyloxyphosphinyl group, and diethoxyphosphinyl group.

The phosphinyloxy group includes, preferably, substituted or non-substituted phosphinyloxy groups of 0 to 30 carbon atoms, for example, diphenylxyphosphinyloxy group and dioctyloxyphosphinyloxy group.

The phosphinylamino group includes, preferably, substituted or non-substituted phosphinylamino groups of 0 to 30 carbon atoms, for example, dimethoxyphosphinylamino group, and dimethylaminophosphinylamino group.

The silyl group includes, preferably, substituted or non-substituted silyl groups of 0 to 30 carbon atoms, trimethylsilyl group, t-butyldimethylsilyl group, and phenyldimethylsilyl group.

Among the substituents described above, those having hydrogen atoms may be substituted at the hydrogen atoms by the substituents described above. Examples of such substituents include, alkylcarbonylaminosulfonyl group, arylcarbonylaminosulfonyl group, alkylsulfonyl aminocarbonyl group, and arylsulfonylaminocarbonyl group. The examples include methylsulfonylaminocarbonyl group, p-methylphenylsulfonylaminocarbonyl group, acetylamino sulfonyl group, and benzoylaminosulfonyl group.

R¹² represented by Formula (I) and Formula (III) represents a substituted or non-substituted aliphatic group, substituted or non-substituted aromatic group, or substituted or non-substituted heterocyclic group. The substituent includes the substituent A described above as an example. R¹² includes, preferably, non-substituted aliphatic groups, more preferably, alkyl groups of 5 or less carbon atoms and, particularly preferably, methyl group or tert-butyl group.

R¹¹ represented by Formula (I) and Formula (II) is a hydroxyl group, carboxyl group, or sulfo-substituted aromatic group (examples including those examples of the aryl groups described for the substituent A. They include preferably a phenyl group or naphthyl group) or heterocyclic group (examples including those examples of the heterocyclic groups described for the substituent A, preferably, pyridyl group, pyrimidyl group, 1,3,5-triazyl group). R¹¹ is, preferably, a heterocyclic group substituted with a hydroxyl group or a carboxyl group and, particularly preferably, hydroxyl group-substituted nitrogen-containing heterocyclic ring.

A particularly preferred structure of the 5-aminopyrazole derivative represented by Formula (I) is a compound represented by Formula (IV).

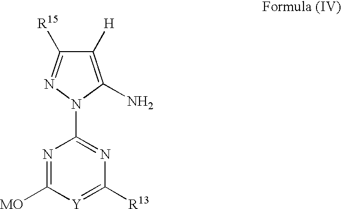

In Formula (IV), R¹³ represents a hydrogen atom or a substituent, Y represents a nitrogen atom or —CR¹⁴ ═, R¹⁵ has the same meanings as those for R¹² of Formula (I), and M represents a hydrogen atom, a metal ion, ammonium, or an organic cation. R¹⁴ represents herein a hydrogen atom or a substituent. In the formula, OM includes forms other than OH (keto-enol tautomerism), which corresponds to “hydroxyl group” in Formula (I).

R¹³ is, preferably, a hydrogen atom, halogen atom, alkyl group, heterocyclic group, hydroxyl group, alkoxy group, amino group, acylamino group, sulfamoylamino group, alkyl or arylsulfonylamino group, mercapto group, alkylthio or arylthio group, and particularly preferably, a hydrogen atom, hydroxyl group, halogen atom, amino group or heterocyclic group of 0 to 8 carbon atoms.

In Formula (IV), Y represents a nitrogen atom or —CR¹⁴═, and Y is preferably a nitrogen atom. In a case where Y is —CR¹⁴═, R¹⁴ is preferably a hydrogen atom, halogen atom, alkyl group of 1 to 8 carbon atoms, alkoxy group of 1 to 8 carbon atoms, or a hydroxyl group.

In Formula (IV), M represents a hydrogen atom, a metal ion, ammonium, or an organic cation and, preferably, a hydrogen atom, an alkali metal ion (for example, lithium ion, sodium ion, potassium ion, cesium ion, etc.), an alkaline earth metal ion (for example, magnesium ion, calcium ion, etc.), silver ion, zinc ion, ammonium, quaternary ammonium (for example, tetraethyl ammonium, tetra-N-heptyl ammonium, dimethylcetyl benzyl ammonium, etc.) or quaternary phosphonium. More preferably, this is a hydrogen atom or an alkali metal ion, or ammonium.

In Formula (I) and Formula (IV), as the substituents for R¹¹ to R¹⁵, the substituent may join to each other to form a bis type, tris type or tetrakis type. R¹³ and R¹⁴ may join to each other to form a cyclic structure.

Then, preferred example of Formula (I) and Formula (IV) are mentioned as specific examples, but the invention is not restricted to them.

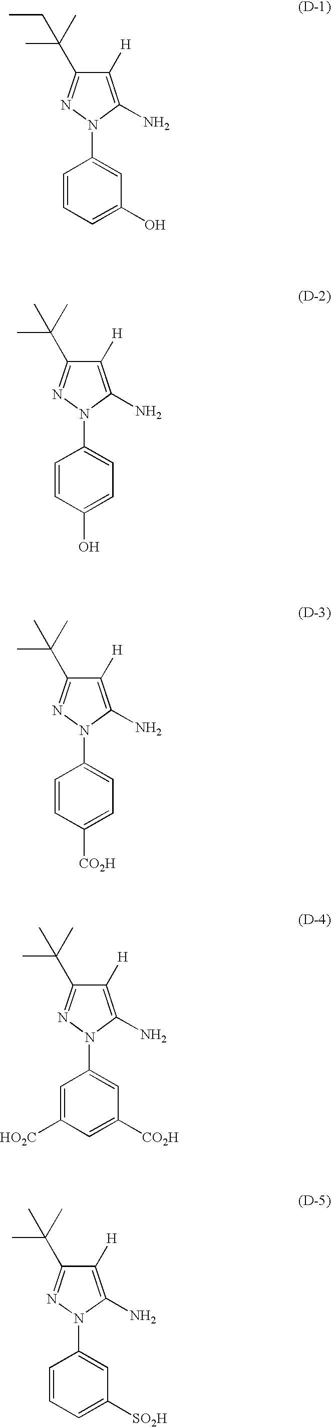

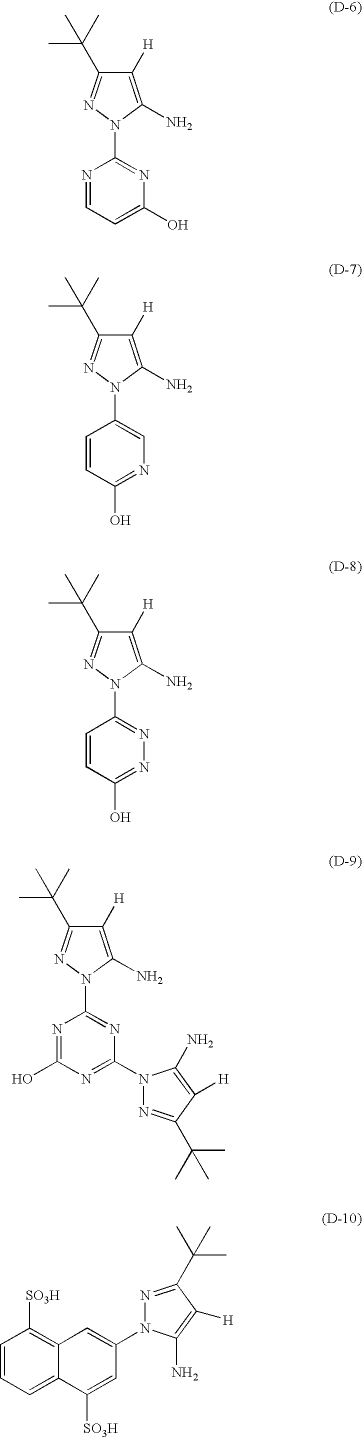

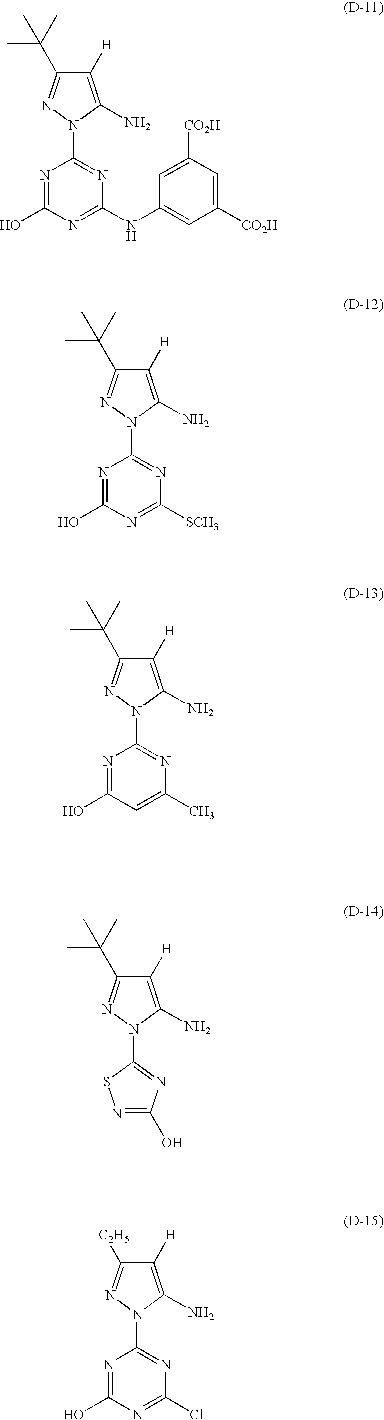

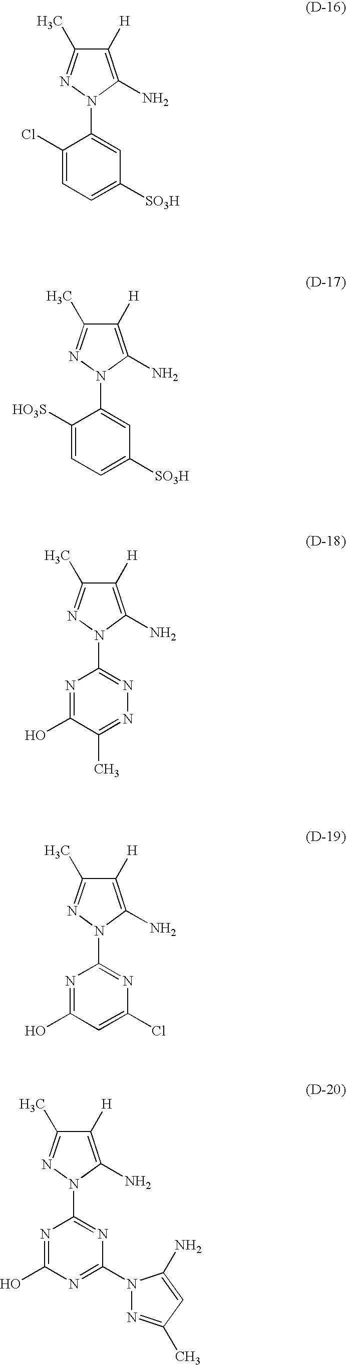

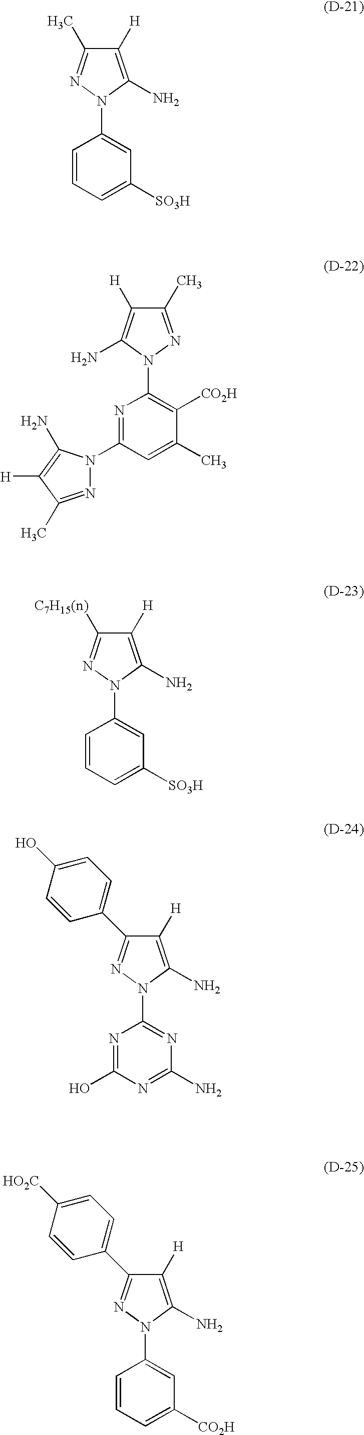

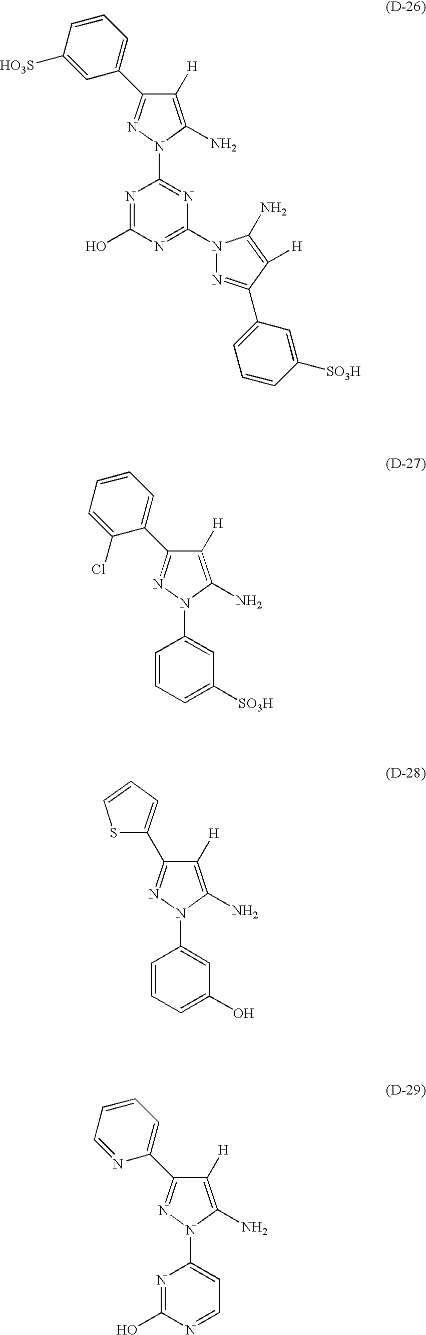

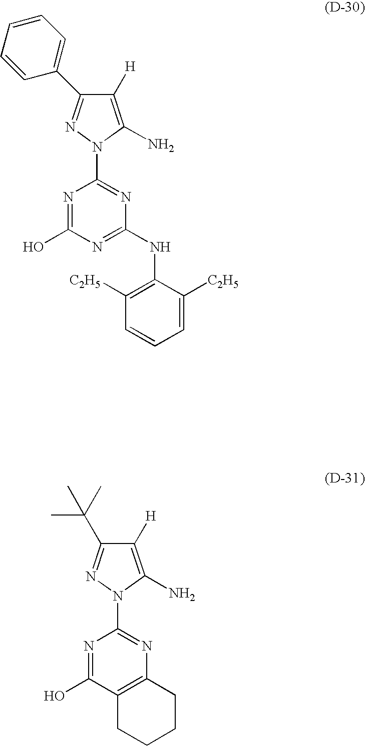

The compound described above of the invention may be present sometimes as a tautomeric isomer depending on the type of the substituent. Both of optional tautomeric isomers in a pure form and an optional mixture of tautomeric isomers are included in the compound of the invention.

Further, in the invention, the compound represented by Formula (I) to Formula (IV) may contain isotopes (for example, ²H, ³H, ¹³C, ¹⁵N) in the structure.

The compound of the invention includes also those containing pair salts depending on the synthesis process or isolation method thereof. The pair salt may be of any type and includes, for example, halide ion, sulfate ion, nitrate ion, carbonate ion, sulfonate ion, phosphate ion, acetate ion, metal ion, and ammonium ion. Depending on the structure, it may form an intra-molecular salt.

Then, a process for producing the 5-aminopyrazole derivative represented by Formula (I) or Formula (IV) from the hydrazine compound represented by Formula (II) and the compound represented by Formula (III) of the invention is to be described specifically.

The production process of the invention has a feature of reacting the hydrazine compound represented by Formula (II) and the compound represented by Formula (III) under the reaction condition satisfying Reaction Condition 1 and the Reaction Condition 2;

Reaction Condition 1: The reaction is conducted in a reaction solvent containing water.

Reaction Condition 2: pH of a liquid mixture containing the hydrazine compound represented by Formula (II), the compound represented by Formula (III), the reaction solvent, and an acidifying agent upon charging is within a range of from 2.0 to 4.0 at 25° C. In the production process of the invention, for adjusting the pH of the liquid mixture upon charging within a range from 2.0 to 4.0, it is necessary to add an acid or alkali. However, in a case where the pH of the liquid mixture of the hydrazine compound represented by Formula (II), the compound represented by Formula (III), and the reaction solvent upon charging is within a range from 2.0 to 4.0 (25° C.), addition of the acid or alkali is not always necessary.

The acidifying agent means an acid for adjusting the pH.

The acid for adjusting the pH includes, for example, inorganic acids such as hydrochloric acid, hydrobromic acid, hydroiodic acid, sulfuric acid, nitric acid, phosphoric acid, and boric acid, or organic acids such as formic acid, acetic acid, trichloroacetic acid, propionic acid, methanesulfonic acid, and p-toluene sulfonic acid. The acid for adjusting the pH is preferably, hydrochloric acid, sulfuric acid, phosphoric acid, or methanesulfonic acid and, particularly preferably, sulfuric acid and hydrochloric acid.

The alkali for adjusting the pH may be an organic base (for example, ammonia, triethylamine, pyridine, etc.) or an inorganic base. The inorganic base used for adjusting the pH includes, for example, sodium hydrogen carbonate, potassium hydrogen carbonate, sodium carbonate, potassium carbonate, sodium acetate, potassium acetate, lithium hydroxide, sodium hydroxide, potassium hydroxide, monohydrogen sodium phosphate, dihydrogen sodium phosphate, and sodium phosphate and the alkali for adjusting the pH is, preferably, sodium hydroxide or potassium hydroxide.

The addition amount of the acid or alkali for adjusting the pH can be selected properly, and it is essential that the pH upon charging a liquid mixture containing the hydrazine compound represented by Formula (II), the compound represented by Formula (III), the reaction solvent, and the acid or alkali is from 2.0 to 4.0 (25° C.) and the pH is preferably from 2.5 to 3.5 (25° C.) and pH is, more preferably, from 2.7 to 3.2 (25° C.).

Further, the pH upon completion of the reaction is preferably within a range from 0.8 to 2.0.

In the production of the compound of Formula (I) or Formula (IV), the reaction proceeds also by using a solvent consisting only of water or a mixed solvent including water and an organic solvent. However, in a case of using a mixed solvent, it is preferred that the water content in the total mass of the reaction solvent is 50 mass % or more, the water content is more preferably, 80 mass % or more and, particularly preferably, this is a solvent consisting only of water in view of production at a low cost.

The type of the organic solvent can be selected properly depending on the type of the reaction system or the like and can include, for example, acetonitrile, propionitrile, butylonitrile, alcohol (for example, methanol, ethanol, i-propylalcohol, ethylene glycol, etc), methylene chloride, chloroform, carbon tetrachloride, dichloroethane, ethyl acetate, butyl acetate, toluene, benzene, hexane, diethyl ether, tetrahydrofuran, 1,4-dioxane, N,N-dimethylformamide (DMF), N,N-dimethylacetoamide (DMAC), N,N-dimethylimidazolidinone (DMI), or sulfolane. The solvents may be combined properly and used as a mixture. The organic solvent in the reaction of the invention is, preferably, acetonitrile or alcohol and, particularly preferably, methanol or ethanol.

Further, while the amount of the solvent to be used is not particularly restricted and can be selected properly in accordance with the type of the reaction system or the like, it is usually about from 1 to 100 times, preferably, from 5 to 50 times and, particularly preferably, from 10 to 30 times by mass ratio of the solvent based on the hydrazine compound of Formula (II).

The reaction temperature in the invention is not particularly restricted and can be selected properly in accordance with the type of the reaction system and the concentration of the compounds of reaction species and it is usually about from 0° C. to 120° C., preferably, from 20° C. to 80° C., more preferably, from 30° C. to 70° C. and, particularly preferably, from 40° C. to 60° C. While also the reaction time is not particularly restricted, it is usually from 30 min to 24 hr, from 2 hr to 15 hr and, particularly preferably, from 4 hr to 12 hr.

The order of adding the hydrazine compound represented by Formula (II), the compound represented by Formula (III), the reaction solvent, and the acid or the alkali into the reaction system is optional and not restricted particularly.

A synthesis scheme of an exemplified compound D-9 as an example of the 5-aminopyrazole derivative substituted with the water-soluble group represented by Formula (I) or Formula (IV) is shown below.

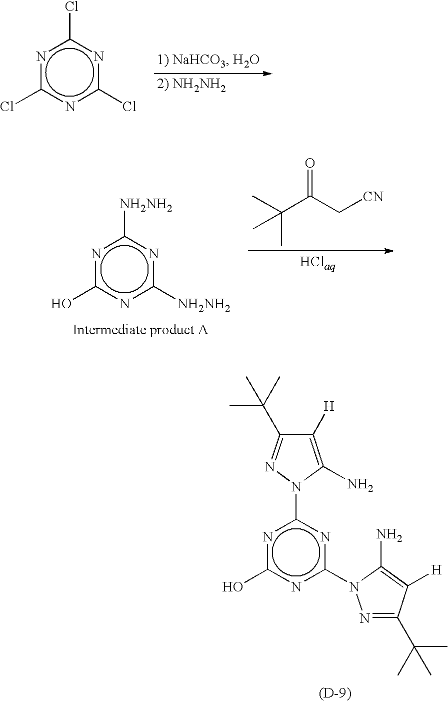

5-aminopyrazole derivative represented by Formula (4) of the invention is to be described specifically.

R²² in Formula (3), Formula (4), and Formula (7) represents an electron-withdrawing group. The electron-withdrawing group is a substituent having an electron-withdrawing property by an electron effect, which is a substituent having a large up value when using the σp value for Hammett's substituent constant as a measure for the electron-withdrawing property or electron donating property of the substituent. The substituent includes, for example, a cyano group, nitro group, halogen atom, sulfo group, trifluoromethyl group, alkoxycarbonyl group, and acyl group.

The σp value for Hammett's substituent constant is to be described briefly. The Hammett's rule is an empirical rule proposed by L. P. Hammett in 1935 for quantitatively discussing the effect of substituents on the reaction or equilibrium of benzene derivatives, the validity of which has been accepted generally at present. The substituent constant of the Hammett's rule includes a σp value and a am value, and such values are found in many general books, and specifically, for example, in “Lange's Handbook of Chemistry” edited by J. A. Dean, 12th edition, 1979 (Mc Graw-Hill), or “Kagaku no Ryoiki”, extra number, 122, pp 96 to 103, 1979 (Nankodo)

R²² in Formula (3), Formula (4), and Formula (7) is preferably electron-withdrawing groups having the up value of 0.2 or more, for example, cyano group, alkyl or arylsulfonyl group, acyl group, carbamoyl group, carboxyl group, and alkoxycarbonyl group and, particularly preferably, a cyano group.

X² represented by Formula (3) represents a halogen atom, hydroxyl group, amino group, alkoxy group, or aryloxy group, X² is preferably a halogen atom or an alkoxy group of 1 to 6 carbon atoms and, particularly preferably, a methoxy group or ethoxy group.

Compounds represented by Formula (3) include the followings.

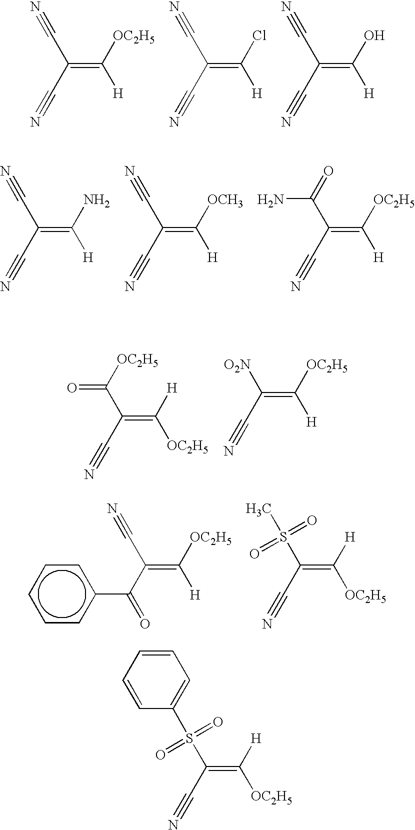

R²¹ in Formula (1), Formula (2), and Formula (4) represents an aromatic group or heterocyclic substituted with a hydroxyl group, carboxyl group, or sulfo group and R²¹ is preferably an aromatic group or heterocyclic group substituted with a carboxyl group or sulfo group and particularly preferred structure as R²¹ is a compound represented by Formula (5).

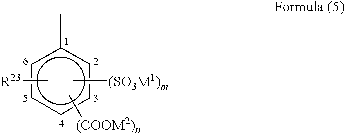

In Formula (5), R²³ represents a hydrogen atom or a substituent, M¹ and M² each represents a hydrogen atom, metal, ammonium or organic cation, m and n each represents an integer of 0 to 3, providing that m+n is an integer of 1 to 3.

R²³ represented by Formula (5) includes a hydrogen atom and the substituent A described above, preferably, a hydrogen atom, an aliphatic group, a halogen atom, an alkoxy group, or a hydroxyl group, more preferably, a hydrogen atom, a halogen atom, alkyl groups of 1 to 8 carbon atoms, alkoxy groups of 1 to 8 carbon atoms, or a hydroxyl group and, particularly preferably, a hydrogen atom or chlorine atom.

M¹ and M² represented by Formula (5) are preferably a hydrogen atom, an alkali metal ion (for example, lithium ion, sodium ion, potassium ion, and cesium ion), an alkaline earth metal ion (for example, magnesium ion, and calcium ion), silver ion, zinc ion, ammonium, quaternary ammonium (for example, tetraethyl ammonium, tetra-N-heptyl ammonium, and dimethylcetyl benzyl ammonium) or quaternary phosphonium. More preferably, they are a hydrogen atom, an alkali metal ion or ammonium.

m in Formula (5) is, preferably, an integer of 0, 1 or 2, n is, preferably, an integer of 0, 1 or 2, m+n is preferably an integer of 1 or 2, more preferably, m=0 and n=2. Formula (5) is particularly preferred in a case where carboxyl groups are substituted on 3-position and 5-position on the benzene ring in Formula (5).

As the heterocyclic group represented by R²¹, the heterocyclic groups in the substituent A described above are applied and examples therefor are also identical.

As the substituents for R²¹, R²², R²³ and X² in Formula (1) to Formula (5), the substituents may join to each other to form a bis type, tri type, or tetrakis type. R²² and X² may join to each other to form a cyclic structure.

Preferred examples of Formula (4) are to be mentioned as specific example but the invention is not restricted to them.

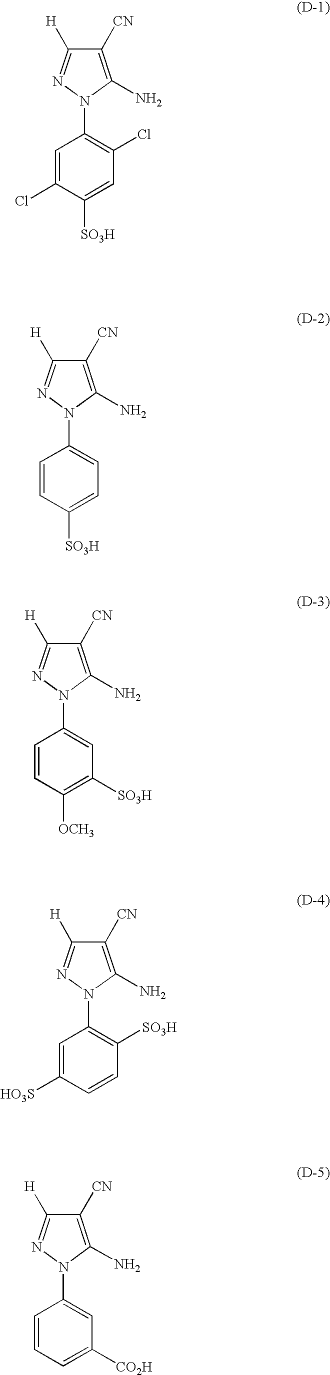

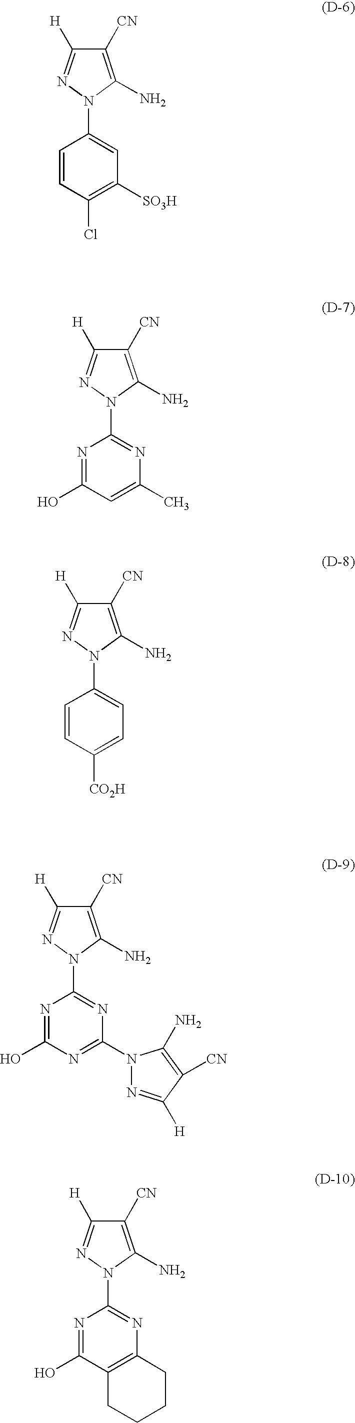

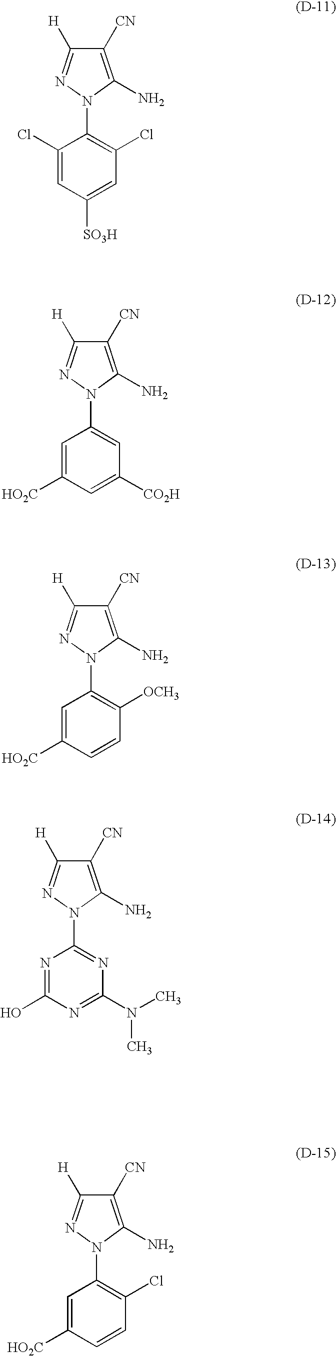

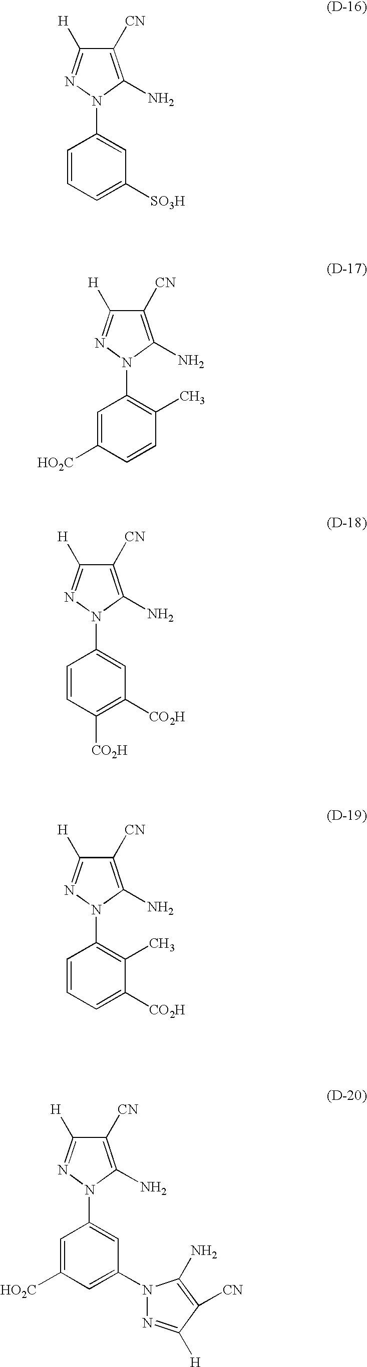

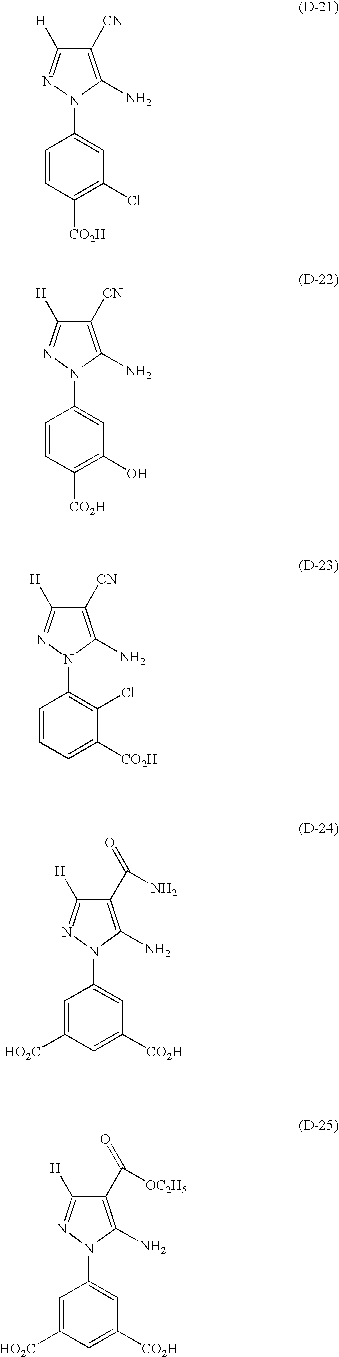

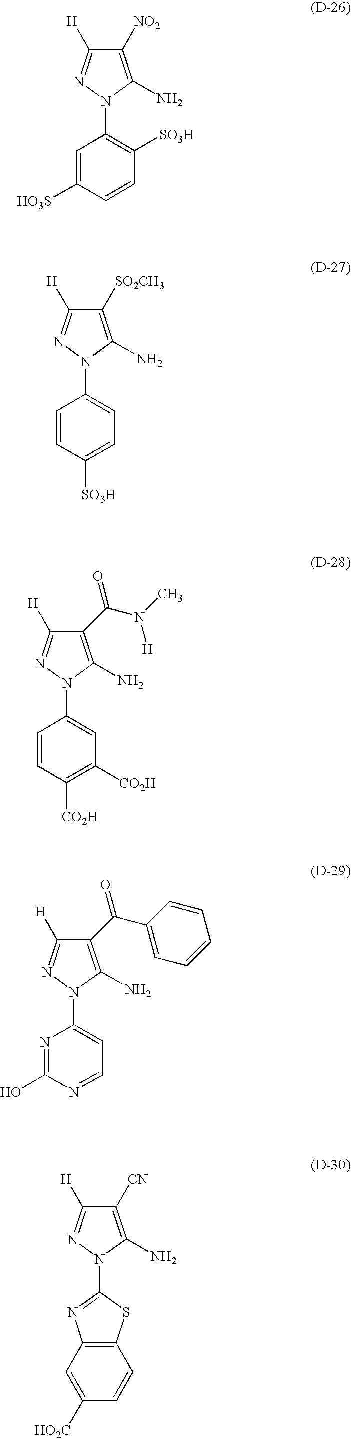

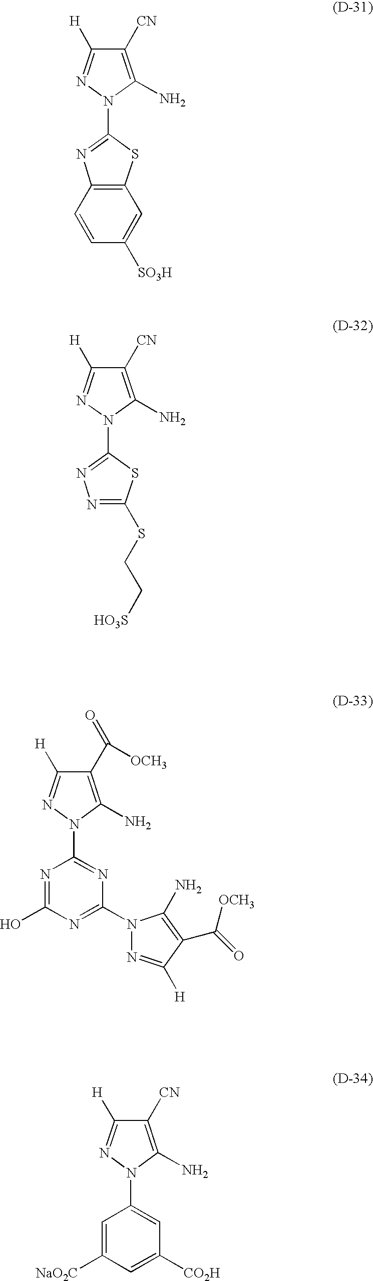

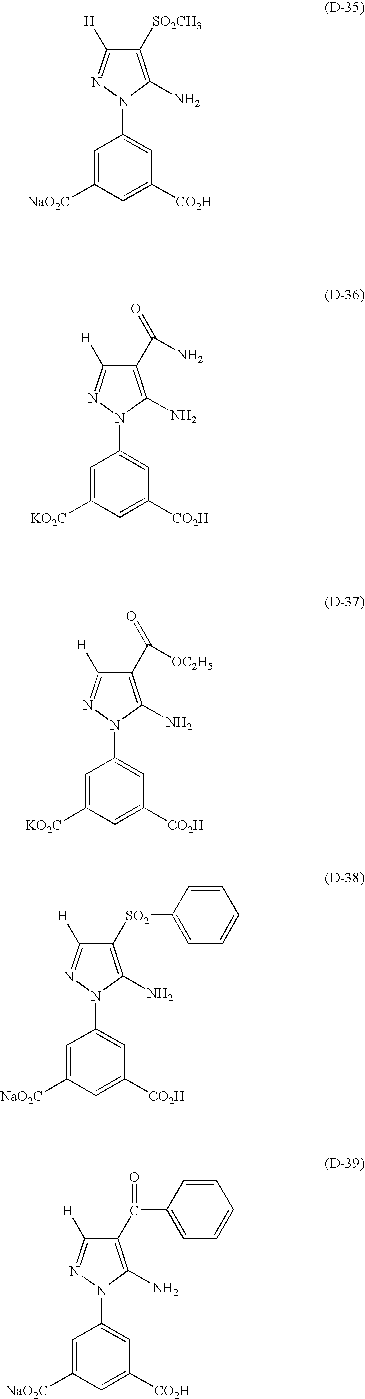

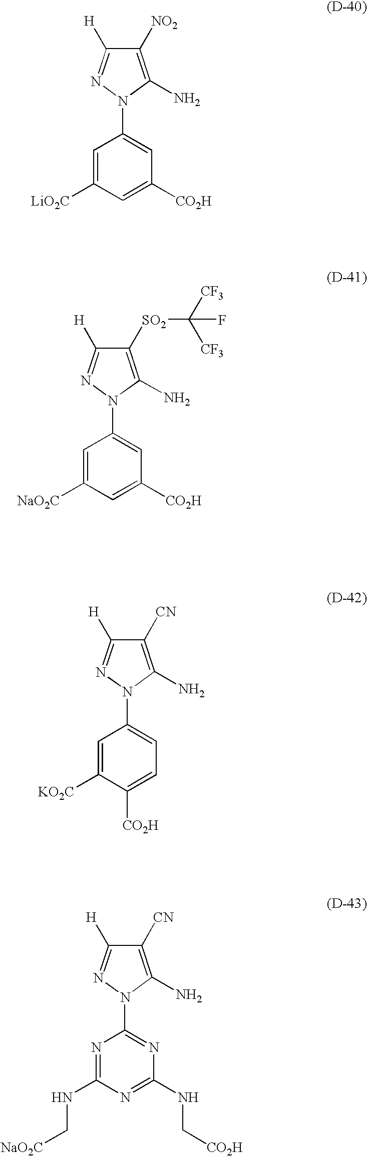

The compound described above of the invention may be present as a tautomeric isomer depending on the type of the substituent. Both of optional tautomeric isomers in a pure form and an optional mixture of tautomeric isomers are included in the compound of the invention.

Further, in the invention, the compound represented by Formula (1) to Formula (5) may contain isotopes (for example, ²H, ³H, ¹³C, ¹⁵N) in the structure.

The compound of the invention includes also those containing pair salts depending on the synthesis process or isolation method thereof. The pair salt may be of any type and includes, for example, halide ion, sulfate ion, nitrate ion, carbonate ion, sulfonate ion, phosphate ion, acetate ion, metal ion, and ammonium ion. Depending on the structure, it may form an intra-molecular salt.

Successively, Formula (6) is to be described.

X² in Formula (6) has the same meanings as those for the description and preferred examples of X² in Formula (3) are described above.

R²⁴ and R²⁵ in Formula (6) each represent independently a group leaving easily by the reaction of Formula (6) and Formula (7).

The leaving groups mean such substituents that leave from the carbon atoms substituted by R²⁴, R²⁵, and X², by the formation of a single covalent bond formed by the nucleophilic reaction of Formula (7) to Formula (6). Generally, such groups are referred to as leaving groups. R²⁴ or ^{R25} accepts a proton to form a stable nonion. When the reaction proceeds further, the previous covalent single bond is induced to a double bond and, correspondingly, one of R²⁴ or ^{R25} remaining in the previous elimination is excluded from the molecule accompanying the proton in the molecule. The two types of the reactions do not sometime proceed or take a time for the proceeding depending on the leaving property of the leaving group. That is, the reaction of Formula (6) and Formula (7) is controlled by the leaving property of the leaving groups R²⁴ and R²⁵ and the reaction proceeds more easily as the leaving of the group is easier.

Preferred examples for R²⁴ and R²⁵ are those each representing independently a halogen atom, hydroxyl group, amino group, alkoxy group, aryloxy group, alkylsulfonyloxy group or arylsulfonyloxy group, more preferably, alkoxy groups of 1 to 6 carbon atoms, alkylsulfonyloxy groups of 1 to 6 carbon atoms, or arylsulfonyloxy groups of 6 to 12 carbon atoms and, particularly preferably, methoxy group or ethoxy group. The halogen atom, hydroxyl group, amino group, alkoxy group, aryloxy group, and the alkyl sulfonyl or arylsulfonyl in the alkylsulfonyloxy group or arylsulfonyloxy group, include those examples and preferred groups described for the substituent A.

The compound represented by Formula (6) includes the followings.

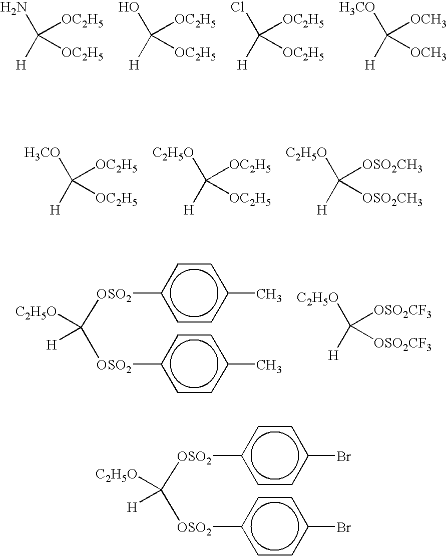

Then, Formula (7) is to be described.

R²² in Formula (7) has the same meanings as descriptions and preferred examples for R²² in Formula (3) and Formula (4) described above.

The compound represented by Formula (7) includes the followings.

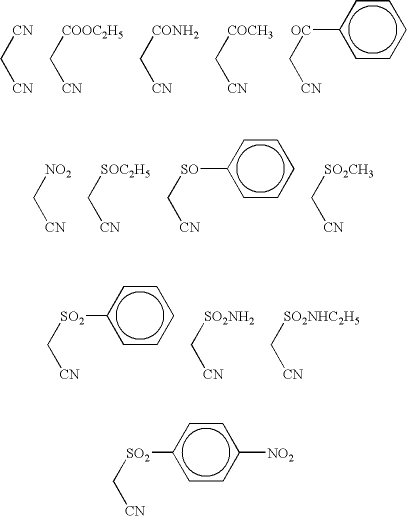

A production process for the 5-aminopyrazole derivative represented by Formula (4) of the invention is to be described specifically.

The production process of the invention has a feature of continuously conducting a step (A) of diazotizing the amine compound represented by Formula (1) to form a diazonium salt, a step (B) of reducing the diazonium salt to form a hydrazine compound represented by Formula (2), and a step (C) of reacting the hydrazine compound with the compound represented by Formula (3) to form a 5-aminopyrazole derivative represented by Formula (4).

The “continuously” means that the reaction is conducted in the same reaction solution without extracting the compounds (intermediates) prepared at each of the steps.

In the production for Formula (4), the reaction proceeds also by using a solvent only consisting of water or a mixed solvent of water and an organic solvent. However, in a case of using the mixed solvent, the kind of the organic solvent is identical with the organic solvent usable in the production for Formula (I) or Formula (IV), and a preferred organic solvent therefore is also identical. Further, in a case of using the mixed solvent, it is preferred that the water content in the total mass of the reaction solvent in each of the step (A), the step (B), and the step (C) is, preferably, 50 mass % or more and, more preferably, 80 mass % or more. A solvent consisting only of water is most preferred in view of production at a low cost.

At first, the step (A) is to be described specifically.

In the production process of the invention, a diazotizing agent and an acid are essential when diazotizing the amine compound represented by Formula (1). The diazotizing agent includes, for example, nitrites (for example, lithium nitrite, sodium nitrite, and potassium nitrite), alkyl nitrites (for example, methyl nitrite, ethyl nitride, propyl nitrite, butyl nitride, and isoamyl nitrite), nitrosyl chloride, nitrosyl sulfuric acid, and nitrogen oxides such as dinitrogen trioxide. The diazotizing agent is, preferably, nitrite salts, alkyl nitrites, and nitrosyl sulfuric acid and, particularly preferably, sodium nitrite.

While the amount of the diazotizing agent used for the diazotizing reaction can be selected properly, it is usually within a range from 0.8 to 4.0 mol and, preferably, from 0.9 to 2.0 mol based on the amine compound represented by Formula (1). It is particularly preferably within a range from 1.0 to 1.2 mol. After the completion of the diazotizing reaction, an excess diazotizing agent may be deactivated. The deactivating agent used herein includes, for example, amidosulfuric acid or urea.

The acid used for the diazotizing reaction includes, for example, inorganic acids such as hydrochloric acid, hydrobromic acid, sulfuric acid, and phosphoric acid, or organic acids such as formic acid, acetic acid, propionic acid, methanesulfonic acid, and p-toluene sulfonic acid. It is particularly preferably hydrochloric acid or sulfuric acid.

While the amount of the acid used for the diazotizing reaction can be selected properly, it is usually within a range from 1 to 15 mol, preferably, from 1.5 to 5.0 and, particularly preferably, from 2.0 to 2.5 mol based on the amine compound represented by Formula (1). In a case where the amount of use is within the range described above, the stability of the formed diazonium salt is preferred.

The solvent in the diazotizing reaction is not restricted so long as it is inert to the reaction and includes, for example, organic acids such as formic acid, acetic acid, and propionic acid, water, and hydrophilic organic solvents such as acetone, methylethylketone, acetonitrile, propionitrile, butylonitrile, alcohol (for example, methanol, ethanol, isopropylalcohol), tetrahydrofurane, 1,4-dioxane, N,N-dimethylformamide (DMF), N,N-dimethylacetoamide (DMAC), N,N-dimethylimidazolidinone (DMI), sulfolane, dimethylsulfoxide (DMSO), and N-methylpyrrolidone (NMP). Further, such solvents may be used in admixture. The solvent in the diazotizing reaction is, preferably, a solvent consisting only of water or a mixed solvent of water and a hydrophilic organic solvent and, particularly preferably, a solvent consisting only of water.

While the amount of the solvent used for the diazotizing reaction is not particularly restricted, it is properly decided depending on the reaction rate, the working property, the economic property, etc.

While the reaction temperature for the diazotizing reaction is not particularly restricted and can be selected properly, for example, in accordance with the kind of the reaction system, the concentration of the compounds of the reaction species, it is usually selected from a range from about −10° C. to about 50° C., and, preferably, from about 0° C. to about 20° C. In a case where the reaction temperature is in the range described above, the stability of the diazonium salt is satisfactory.

While the reaction time of the diazotizing reaction is not defined generally since it is different depending on the amount of the reaction reagents and the reaction temperature, it may be selected usually from a range from about 10 min to about 24 hr.

In the diazotizing reaction, the order of adding the amine compound represented by Formula (1), the diazotizing agent, the acid, and the solvent into the reaction system is optional and not restricted particularly.

Then, the step (B) is to be described specifically.

In the production process of the invention, a reducing agents is essential upon reducing the diazonium salt. The reducing agent includes, for example, those reducing agent described in New Experimental Chemistry Session”, Vol 14, pp 1580, published in 1978, from Maruzen Co., or “Experimental Chemical Session”, Vol 20, pp 338, published from Maruzen Co. in 1992. The reducing agent is preferably stannic chloride (II), sulfites, hydrogen sulfites, disulfite, etc. and, particularly preferably, sodium sulfite or sodium disulfite.

The addition amount of the reducing agent used for the reducing reaction can be selected properly and it is usually within a range from 0.5 to 20 mol, preferably, from 1.0 to 10 mol and, particularly preferably, from 1.2 to 3.0 mol based on the amine compound represented by Formula (I).

The reaction temperature for the reducing reaction is not particularly restricted and can be selected properly in accordance with the type of the reaction system or the concentration of the compounds of reaction species, it is usually selected from a range from about 10° C. to about 100° C. and, preferably, about from 10° C. to about 60° C.

While the reaction time for the reducing reaction is not generally defined since this is different depending on the addition amount of the reducing agent and the reaction temperature, it may be selected usually within a range from about 10 min to about 24 hr.

In the reducing reaction, while the order of adding the reducing agent and the diazonium salt formed in the step of (A) into the reaction system is optional and not restricted particularly, an order of adding the diazonium salt formed in the step (A) to the reducing agent is preferred. In a case of adding the diazonium salt to the reducing agent, a solvent is used preferably for dissolving or suspending the reducing agent. The solvent for dissolving or suspending the reducing agent is not restricted so long as it is inert to the reaction and includes, for example, those solvents used for the diazotizing reaction described above. The solvent for dissolving or suspending the reducing agent is, preferably, water or a mixed solvent of water and a hydrophilic organic solvent, and, particularly preferably, a solvent consisting only of water.

In a case of using the sulfite, hydrogen sulfite, or disulfite as the reducing agent in the reducing reaction, it is necessary to conduct acidic hydrolysis in order to obtain the hydrazine compound represented by Formula (2). The acid used for the acidic hydrolyzing reaction includes, for example, those acids used for the diazotizing reaction described above. The acid used for the hydrolytic reaction is, preferably, hydrogen chloride or sulfuric acid.

The addition amount of the acid used for the acidic hydrolyzing reaction can be selected properly but it is usually within a range from 0.5 to 10 mol, preferably, from 1 to 5.0 mol and, particularly preferably, from 1.5 to 3.0 mol based on the amine compound represented by Formula (1).

While the reaction temperature for the acidic hydrolyzing reaction is not particularly restricted and can be selected properly in accordance with the type of the reaction system for the concentration of the compounds of reaction species, it is usually selected within a range from about 10° C. to about 120° C. and, preferably, from about 50° C. to about 90° C.

Since the reaction time for the acidic hydrolyzing reaction is different depending of the addition amount of the acid and the reaction temperature, it is not defined generally. However, the time may usually be selected from a range from about 10 min to about 24 hr.

Then, the step (C) is to be described specifically.

In the production process of the invention, while the addition amount of the compound represented by Formula (3) can be selected properly, it is usually within a range from 0.70 to 5.0 mol, preferably, from 0.80 to 2.5 mol and, particularly preferably, from 0.85 to 1.5 mol based on the amine compound represented by Formula (1).

While the reaction temperature for the step (C) is not particularly restricted and can be selected properly in accordance with the type of the reaction system or the concentration of compounds of reaction species, it is usually selected from a range from about 10° C. to about 120° C., preferably, from about 30° C. to about 70° C. Since the reaction time for the step (C) is different depending on the addition amount of the compound represented by Formula (3) or the reaction temperature, it is not defined generally but may be selected within a range usually from about 10 min to about 24 hr.

In the step (C), the order of adding the compound represented by Formula (3) and the hydrazine compound formed in the step (B) into the reaction system is optional and not restricted particularly. However, it is preferred to add an alkali reagent to the hydrazine compound formed in the step (B) to adjust the pH within a range from 5.0 to 9.0 and then add the compound represented by Formula (3) into the reaction system. A particularly preferred pH is within a range from 5.5 to 7.5.

The alkali reagent used for adjusting the pH of the reaction solution may be either an organic base (for example, ammonia, triethylamine, and pyridine) or an inorganic base. The inorganic base used for adjusting the pH includes, for example, sodium hydrogen carbonate, potassium hydrogen carbonate, sodium carbonate, potassium carbonate, sodium acetate, potassium acetate, lithium hydroxide, sodium hydroxide, potassium hydroxide, more preferably, sodium hydroxide and potassium hydroxide and, particularly preferably, sodium hydroxide.

Then, a method of taking out the 5-aminopyrazole derivative formed in the step (C) from the inside of the reaction system is to be described specifically.

The method of taking out the 5-aminopyrazole derivative represented by Formula (4) formed in the step of (C) from the inside of the reaction system includes crystallizing filtration or separating extraction but is not restricted particularly. The method of taking out the 5-aminopyrazole derivative from the inside of the reaction system includes, preferably, a method of adjusting the pH of the mixed reaction solution containing the formed 5-aminopyrazole derivative to 0.1 to 4.0 and filtering the precipitated crystals. A particularly preferred pH for the reaction mixture is from 0.5 to 2.0, at which the 5-aminopyrazole derivative can be obtained with good filterability and at a high yield and a high purity.

The acidifying agent for controlling the pH includes, for example, those acids used for the diazotization in the step (A). The acidifying agent is, preferably, sulfuric acid or hydrochloric acid and, particularly preferably, sulfuric acid.

In the production process of the invention, the amount of the inorganic salt formed in the neutralizing operation with the alkali reagent in the step (C) can be decreased by decreasing the amount of the acid used for the diazotizing reaction in the step (A) and the amount of the reducing agent used for the reducing reaction in the step (B), and the amount of the acid used in the acidic hydrolysis. In a case where the acid or the reducing agent are used within the particularly preferred ranges described above, since the amount of the inorganic acid to be formed is also small, reaction is possible at a high concentration which is preferred also in view of the improvement for the productivity.

Successively, the reaction step for producing the compound of Formula (3) from the compounds of Formula (6) and Formula (7) is to be described.

The ratio (molar ratio) of the compound represented by Formula (6)/the compound represented by Formula (7) to be used in the reaction of Formula (6) and Formula (7) is preferably 1/3 to 10/1, more preferably, 1/2 to 5/1 and, further preferably, 1/1 to 2/1.

The solvent in the reaction of Formula (6) and Formula (7) is not restricted so long as it is inert to the reaction and includes, for example, organic acids such as formic acid, acetic acid, and propionic acid, acid anhydrides such as acetic acid anhydride and butyric acid anhydride, water, acetone, methyl ethyl keton, acetonitrile, propionitrile, butylonitrile, alcohol (for example, methanol, ethanol, and isopropyl alcohol), acetate esters such as ethyl acetate or butyl acetate, or hydrophilic organic solvents such as tetrahydrofuran, 1,4-dioxane, N,N-dimethylformamide (DMF), N,N-dimethylacetoamide (DMAC), N,N-dimethylimidazolidinone (DMI), sulfolane, dimethylsulfoxide (DMSO), N-methylpyrrolidone (MNP). Further, such solvents may be used in admixture. The solvent is preferably alcohol, organic acid, acid anhydride, acetonitrile or acetone and, particularly preferably, methanol, isopropanol, acetone, acetonitrile, acetic acid, acetic acid anhydride, DMF or DMAC. The amount of the solvent to Formula (7) is 100 times or less, more preferably, 50 times or less and, further preferably, 10 times or less. In a case where it is 100 times or more, the productivity is poor and this is not economical.

The reaction for Formula (6) and Formula (7) can be attained with no solvent (neat). In this case, an acid, and an additive may be used together. The base includes organic bases such as triethylamine, tributylamine, diisopropylethylamine, pyridine, dimethylaminopyridine, DBU (1,8-azabicyclo[5.4.0]-7-undecene), and inorganic bases such as lithium carbonate, sodium carbonate, potassium carbonate, sodium hydrogen carbonate, lithium hydroxide, sodium hydroxide, and potassium hydroxide. Further salts of organic acids and strong bases include lithium acetate, potassium acetate, sodium oxalate, and ethylene diamine disodium tetraacetate. Preferred are lithium hydroxide, sodium hydroxide, potassium hydroxide, lithium acetate, sodium acetate, potassium acetate, triethylamine or pyridine. More preferred are lithium hydroxide, potassium hydroxide, lithium acetate, and potassium acetate.

The amount of the base to Formula (7) is from 0 to 5 times molar ratio and, more preferably, from 0 to 2 times and, further preferably, from 0 to 1.2 times.

As the acid, an inorganic acid (also referred to as mineral acid) and an organic acid can be used. The inorganic acid includes hydrochloric acid, phosphoric acid, and sulfuric acid and, preferably, phosphoric acid and sulfuric acid and, further preferably, sulfuric acid. The organic acid includes formic acid, acetic acid, propionic acid, and methansulfonic acid, preferably, acetic acid, propionic acid and methanesulfonic acid, and further preferably, methanesulfonic acid. Further, the acids may be used each alone or may be used in admixture.

The amount of the acid to Formula (7) as the molar ratio is from 0 to 5 times, more preferably, from 0 to 3 times and, further preferably, from 0 to 2 times.

As the effect of the additive, by-products due to the reaction of Formula (6) and Formula (7) can be transformed by the chemical reaction to another molecules. This can induce the chemical equilibrium to the reaction side of the aimed product. Such additives include anhydrides. Preferred examples are acetic acid anhydride, phthalic acid anhydride, and benzoic acid anhydride, more preferably, acetic acid anhydride and phthalic acid anhydride and, further preferably, acetic acid anhydride.

The amount of the additives to Formula (7), based on the molar ratio, is from 0 to 5 times, more preferably, from 1 to 4 times and, further preferably, from 1 to 3 times.

The reaction temperature is, preferably, from 70° C. to 120° C., more preferably, from 80° C. to 110° C. and, further preferably, from 85° C. to 105° C. In a case where the reaction temperature is lower than 70° C., the reaction is retarded to take a long time, which is not economical. Further, in a case where it is higher than 120° C., it is not preferred since there may be a possibility that the starting materials are decomposed (for example, the compound represented by Formula (6) or the aimed compound represented by Formula (3)).

Further, at the reaction temperature within the range described above, by-products accompanying the reaction can be distilled off at an ambient pressure to provide an advantage capable of simplifying the steps compared with the procedure of distilling off the by-products under a reduced pressure or the ambient pressure after the reaction.

The reaction temperature is, preferably, from 30 to 6 hr, more preferably, from 1 hr to 5 hr, and further preferably, from 2 hr to 5 hr. In a case where the reaction time is less than 30 min, it is considered that the reaction is not completed. Further, in a case where the reaction time is 6 hr or more, it may be a possibility that the compound represented by Formula (3) is decomposed.

The step (C) in a case of using the reaction mixture of the compounds represented by Formula (3) obtained in the reaction step described above is to be described specifically.

The addition amount of the reaction mixture in the production process of the invention is within such a range that the amount of the compound represented by Formula contained in the reaction mixture to the amine compound represented by Formula (1) is within a range, preferably, from 0.70 to 5.0 mol, more preferably, from 0.80 to 2.5 mol and, particularly preferably, from 0.85 to 1.5 mol. The reaction temperature for the step (C) is not particularly restricted and can be selected properly in accordance with the type of the reaction system or the concentration of the compound of the reaction species but it is selected usually from a range from about 10° C. to about 120° C. and, preferably, from about 30° C. to about 70° C. While the reaction time for the step (C) is not defined generally since it is different depending on the addition amount of the reaction mixture and the reaction temperature, it may be usually selected within a range from about 10 min to about 24 hr.

In the step of (C), the order of adding the reaction mixture containing the compound represented by Formula (3) by the reaction of the compound represented by Formula (6) and the compound represented by Formula (7), and the hydrazine compound formed in the step (B) is optional and not restricted particularly. However, it is preferred to add an alkali reagent to the hydrazine compound formed in the step (B) to adjust the pH within a range from 5.0 to 9.0 and then add the compound represented by Formula (3) into the reaction system. In this case, in a case of using the reaction mixture containing the compound represented by Formula (3), pH fluctuation may occur due to the ingredients in the reaction mixture. Accordingly, the reaction per se proceeds within a range lower than the range determined as described above. From the foregoings, pH for the reaction step (C) is within a range, preferably, from 3.0 to 9.0 and, more preferably, from 3.5 to 8.5 and, further preferably, within range from 3.5 to 8.0. Although depending on the concentration of the reactants contained in the reaction system, in a case where the pH is lower than 3.0, the compound represented by Formula (2) precipitates as an acid salt to inhibit the proceeding of the reaction. Further, in a case where the pH region of the reaction exceeds pH 9.0, the compound represented by Formula (3) is decomposed and the aimed reaction does not proceed quantitatively.

The alkali regent used for adjusting the pH of the reaction solution may be either organic bases (for example, ammonia, triethylamine, pyridine, etc.) or inorganic bases. The inorganic bases used for adjusting the pH is, for example, sodium hydrogen carbonate, potassium hydrogen carbonate, sodium carbonate, potassium carbonate, sodium acetate, potassium acetate, lithium hydroxide, sodium hydroxide, and potassium hydroxide, more preferably, sodium hydroxide, and potassium hydroxide and, particularly preferably, sodium hydroxide.

Then, a method of taking out the 5-aminopyrazole derivative formed in the step (C) from the inside of the reaction system in a case of using the reaction mixture of the compound represented by Formula (3) is to be described specifically. Since pH is set low as described above for the 5-aminopyrazole derivative represented by Formula (4) formed in the step (C), the derivative is precipitated from the inside of the reaction system. pH upon precipitation is different depending on the concentration of the reactants and the products contained in the reaction system but pH is within a range from 3.0 to 5.0. pH is, preferably, within a range from 3.5 to 5.0 and, more preferably, pH is within a range from 4.0 to 5.0. In a case where the pH is lower than 3.0, precipitated crystals are swollen to worsen the stirring performance. In a case where pH is higher than 5.0, precipitation is worsened to cause lowering of the yield. Further, an acid may be dropped till the range of the pH described above. Those acids described for the reaction step of the compound of Formula (3) can be mentioned. While preferred examples of the acid are also identical, acetic acid is particularly preferred. The taking out method includes, for example, crystallizing filtration or separating extraction with no particular restriction. The method of taking out the 5-aminopyrazole derivative from the inside of the reaction system includes, preferably, a method of filtering the precipitated 5-aminopyrazole derivative crystallized by the reaction as it is.

Successively, description is to be made specifically for an azo dye in a case of using the 5-aminopyrazole derivative obtained by the production process of the invention as an intermediate dye product.

The azo dye in a case of using the 5-aminopyrazole derivative obtained by the production process according to the invention as a dye intermediate product is an azo dye represented by Formula (a) produced by using a 5-aminopyrazole derivative (coupler component) represented by Formula (I) produced by the production process described in 1 to 6 above, and an azo dye represented by Formula (b) produced by using a 5-aminopyrazole derivative (diazo component) represented by Formula (4) produced by the production process as described in 7 to 16 above.

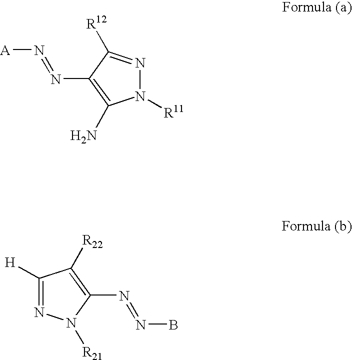

A in Formula (a) and B in Formula (b) represent each independently a mononuclear ring or a condensed ring which may have a substituent. The ring described above includes aromatic hydrocarbon rings, non-aromatic hydrocarbon rings, aromatic heterocyclic rings, or non-aromatic heterocyclic rings. Among them, the aromatic heterocyclic rings or non-aromatic heterocyclic rings are preferred, the aromatic heterocyclic rings are more preferred, and nitrogen-containing 5- to 7-membered aromatic heterocylic rings are most preferred.

The mononuclear ring or the condensed ring represented by A or B includes, for example, benzene ring, imidazole ring, benzoimidazole ring, pyrazole ring, benzopyrazole ring, triazole ring, thiazole ring, benzothiazole ring, isothiazole ring, benzoisothiazole ring, oxazole ring, benzooxazole ring, thiadiazole ring, pyrrol ring, benzopyrrol ring, indole ring, isooxazole ring, benzoisooxazole ring, thiophene ring, benzothiophene ring, furan ring, pyran ring, benzofuran ring, pyridine ring, quinoline ring, isoquinoline ring, pyridazine ring, pyrimidine ring, pyrazine ring, cinnoline ring, phthtalazine ring, quinazoline ring, quinoxaline ring, or triazine ring. The mononuclear ring or the condensed ring represented by A or B is, preferably, imidazole ring, pyrazole ring, triazole ring, thiazole ring, isothiazole ring, and thiadiazole ring, and, particularly preferably, pyrazole ring, isothiazole ring, and thiadiazole ring.

The azo dye in a case of using the 5-aminopyrazole derivative obtained by the production process according to the invention as the dye intermediate product is, most preferably, an azo dye represented by Formula (c) which is produced by using the 5-aminopyrazole derivative represented by Formula (1) (coupler component) produced by the production process described in 1 to 6 above, and the 5-aminopyrazole derivative represented by Formula (4) (diazo component) produced by the production process described in 7 to 16 above.

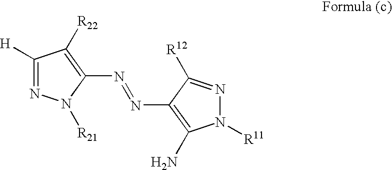

Various methods have been known long since for the synthesis method of azo compounds and synthesis by oxidizing reaction, synthesis by reducing reaction, synthesis by substitution reaction, synthesis by addition reaction, synthesis by condensating reaction and other synthesis methods are known, for example, as described in New Experimental Chemistry Session, Vol. 14-III, pp 1516 to 1534 (Maruzen Co.). Among them, a synthesis method of an azo compound by a diazotizing coupling reaction is a most general method.

The azo dye represented by Formula (a) can be synthesized by a diazotizing coupling reaction of A-NH₂ as a diazo component and the 5-aminopyrazole derivative represented by Formula (I) obtained by the production process according to the invention as a coupler component.

The diazo component A-NH₂ in the diazotizing coupling reaction is not particularly restricted so long as the diazonium salt of A-NH₂ and the 5-aminopyrazole derivative represented by Formula (I) are reacted to exhibit a color.

The azo dye represented by Formula (b) can be synthesized by a diazotizing coupling reaction of the 5-aminopyrazole derivative represented by Formula (4) obtained by the production process of the invention as a diazo component and B as a coupler component.

The coupler component B in the diazotizing coupling reaction is not particularly restricted so long as it reacts with the diazonium salt of the 5-aminopyrazole derivative represented by Formula (4) obtained by the production process of the invention, and then provides a dye showing a color. Referring specifically to the coupler components, they are organic compounds having at least amino group or hydroxyl group and having a reaction site with a diazonium salt. Specifically, usable compounds mean amino compounds or hydroxyl compounds having rings which may have substituents, and also includes mono-rings and condensed rings. The rings include aromatic hydrocarbon rings, non-aromatic hydrocarbon rings, aromatic heterocyclic rings, or non-aromatic heterocyclic rings. Among them, aromatic heterocyclic amino compounds and hydroxyl compounds, or non-aromatic heterocyclic amino compounds and hydroxyl compounds are preferred and aromatic heterocyclic amino compounds and hydroxyl compounds are more preferred, and nitrogen-containing 5- to 7-membered aromatic heterocyclic amino compounds and hydroxyl compounds are further preferred. Preferred examples of the ring in the amino compound and the hydroxyl compound having a ring which may have a substituent include benzene ring, imidazole ring, benzoimidazole ring, pyrazole ring, benzopyrazole ring, triazole ring, thiazole ring, benzothiazole ring, isothiazole ring, benzoisothiazole ring, oxazole ring, benzooxazole ring, thiadiazole ring, pyrrol ring, benzopyrrol ring, indole ring, isooxazole ring, benzoisooxazole ring, thiophene ring, benzothiophene ring, furan ring, pyrane ring, benzofuran ring, pyridine ring, quinoline ring, isoquinoline ring, pyridazine ring, pyrimidine ring, pyrazine ring, sinnoline ring, phthaladine ring, quinazoline ring, quinoxaline ring, or triazine ring. However, the position for the reaction site of the amino group and hydroxyl and, further, diazonium salt, the substituents present on the ring and the positions for substitution are not restricted.

More preferred examples of the ring in the amino compounds having the rings are 2-aminopyridine, 3-aminopyridine, 4-aminopyridine, 2-aminopyridine, 4-aminopyridine, 5-aminopyridine, 3-aminopyridazine, 4-aminopyridazine, 2-aminopyrrole, 3-aminopyrrole, 2-aminoimidazole, 4-aminoimidazole, 3-aminopyrazole, 4-aminopyrazole, 5-aminopyrazole, 4-aminotriazole, 4-amino-1,2,3-triazole, 3-amino-1,2,4-triazole, 2-aminooxazole, 4-aminooxazole, 5-aminooxazole, 3-aminoisooxazole, 4-aminoisooxazole, 5-aminoisooxazole, 2-aminothiazole, 4-aminothiazole, 5-aminothiazole, 3-aminoisothiazole, 4-aminoisothiazole, 5-aminoisothiazole, 2-amino 1,3,4-thiadiazole, 3-amino-1,2,4-thiadiazole, or 5-amino-1,2-4-thiadiazole, and more preferred examples are 2-aminopyrrole, 3-aminopyrrole, 3-aminopyrazole, 4-aminopyrazole, 5-aminopyrazole, 2-aminothiazole, 4-aminothiazole, 5-aminothiazole, 2-amino-1,3,4-thiazole, 3-amino-1,2,4-thiadiazole, or 5-amino-1,2,4-thiadiazole. Among them, 5-aminopyrazole, 2-amino-1,3,4-thiazole, or 5-amino-1,2,4-thiadiazole is particularly preferred, and 5-aminopyrazole is most preferred.

In the hydroxyl compounds having the rings, more preferred examples of the rings are 4-phenol, 4-hydroxypyridine, 3-hydroxypyridine, 2-hydroxypyridine, 2-hydroxypyrimidine, 4-hydroxypyrimidine, 5-hydroxypyrimidine, 3-hydroxypyridazine, 4-hydroxypyridazine, 2-hydroxypyrrol, 3-hydroxypyrrol, 2-hydroxyimidazole, 4-hydroxyimidazole, 3-hydroxypyrazole, 4-hydroxypyrazole, 5-hydroxypyrazole, 4-hydroxytriazole, 4-hydroxy-1,2,3-triazole, 3-hydroxy-1,2,4-triazole, 2-hydroxyxazole, 4-hydroxyxazole, 5-hydroxyxazole, 3-hydroxyisooxazole, 4-hydroxyisooxazole, 5-hydroxyisooxazole, 2-hydroxythiazole, 4-hydroxythiazole, 5-hydroxythiazole, 3-hydroxyisothiazole, 4-hydroxyisothiazole, 5-hydroxyisothiazole, 2-hydroxy-1,3,4-thiadiazole, 3-hydroxy-1,2,4-thiadiazole, or 5-hydroxy-1,2,4-thiadiazole. More preferred examples are 2-hydroxypyrrole, 3-hydroxypyrrole, 3-hydroxypyrazole, 4-hydroxypyrazole, 5-hydroxypyrazole, 2-hydroxythiazole, 4-hydroxythiazole, 5-hydroxythiazole, 2-hydroxy-1,3,4-thiadiazole, 3-hydroxy 1,2,4-thiadiazole, or 5-hydroxy-1,2,4-thiadiazole. Among them, 5-hydroxypyrazole, 2-hydroxy-1,3,4-thiadiazole, or 5-hydroxy-1,2,4-thiadiazole are particularly preferred, and 5-hydroxypyrazole is most preferred.

As the spectral absorption characteristics of an azo dye produced from the 5-aminopyrazole derivative according to the production process of the invention, it is desirable that an absorption maximum is present within a range from 400 nm to 500 nm. More preferably, it is within a range from 420 nm to 480 nm, further preferably, from 435 nm to 480 nm and, most preferably, from 435 nm to 470 nm. In a case where the absorption maximum wavelength is less than 400 nm, a color density as the yellowness is worsened and in a case where it is more than 500 nm, redness increases which is not preferred for the yellow hue. The half-value width of the absorption band is desirably from 60 nm to 100 nm. It is, preferably, within a range from 65 nm to 100 nm, more preferably, from 70 nm to 100 nm and, most preferably, from 70 nm to 90 nm. In a case where it is less than 60 nm, it is difficult to reproduce a mixed color with other colors (magenta and cyan). In a case where it exceeds 100 nm, blurred yellow is exhibited, which is not preferred chromatically.

Preferred examples of the azo dyes of Formula (a), Formula (b) and Formula (c) are to be mentioned as specific example but the invention is not restricted to them.

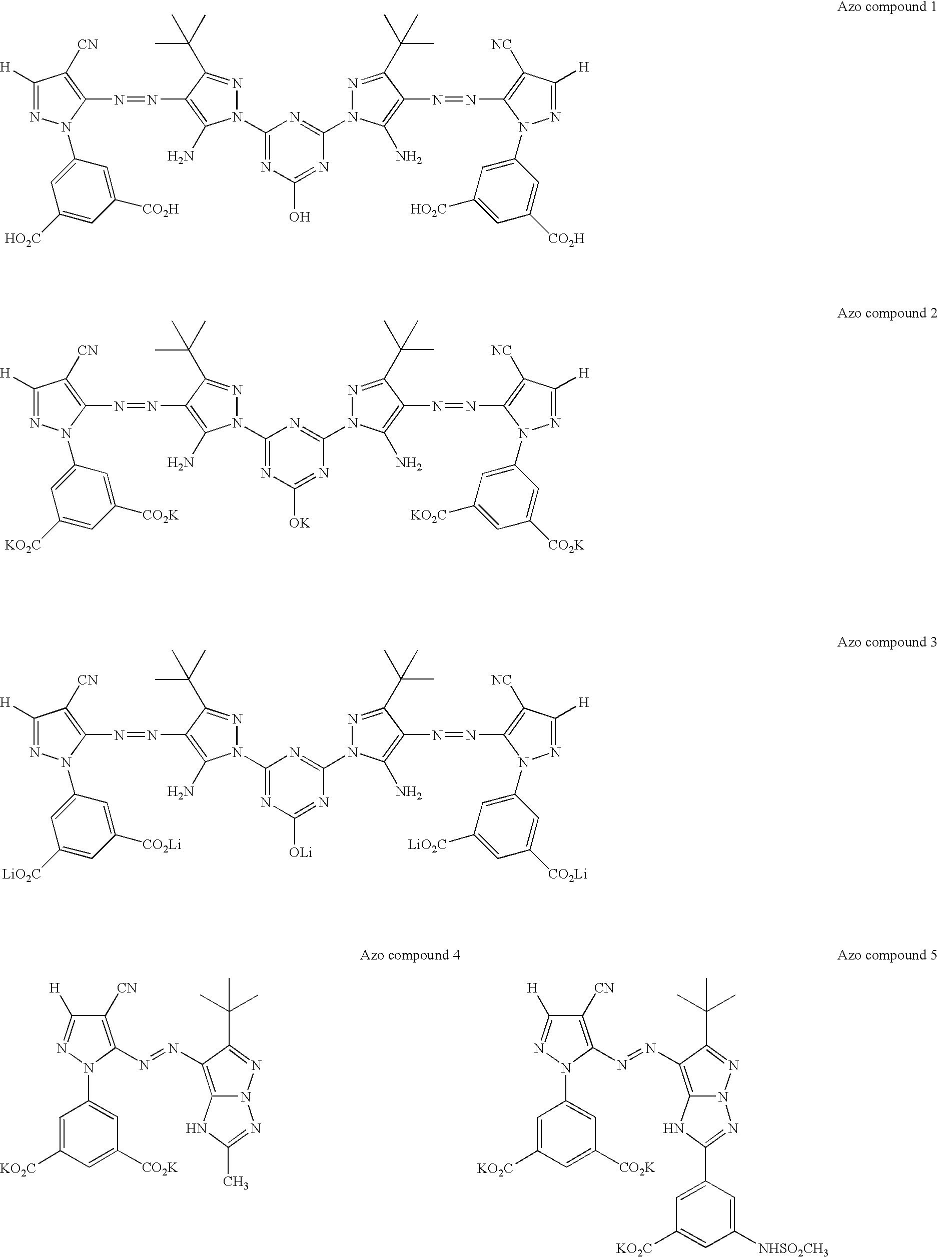

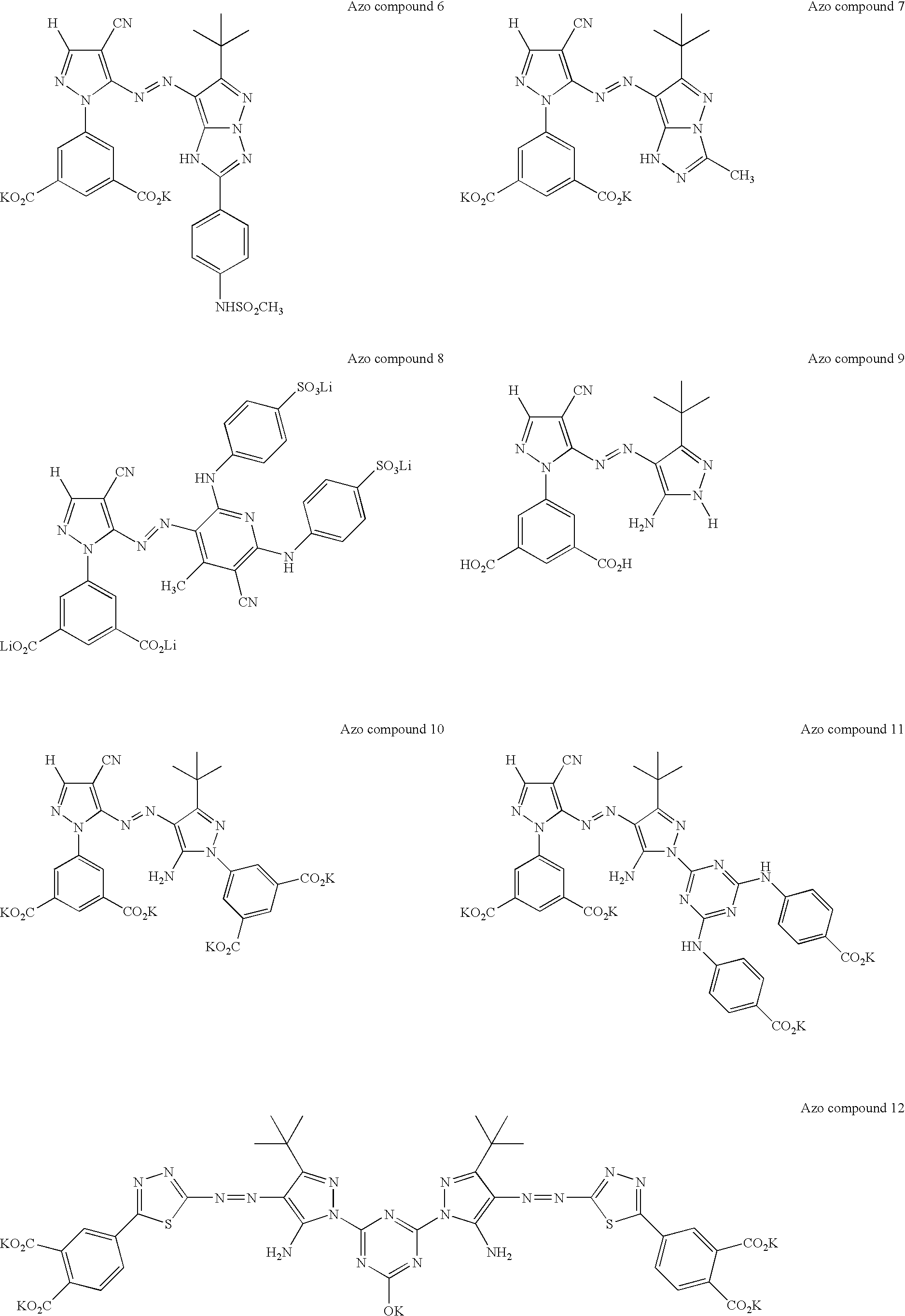

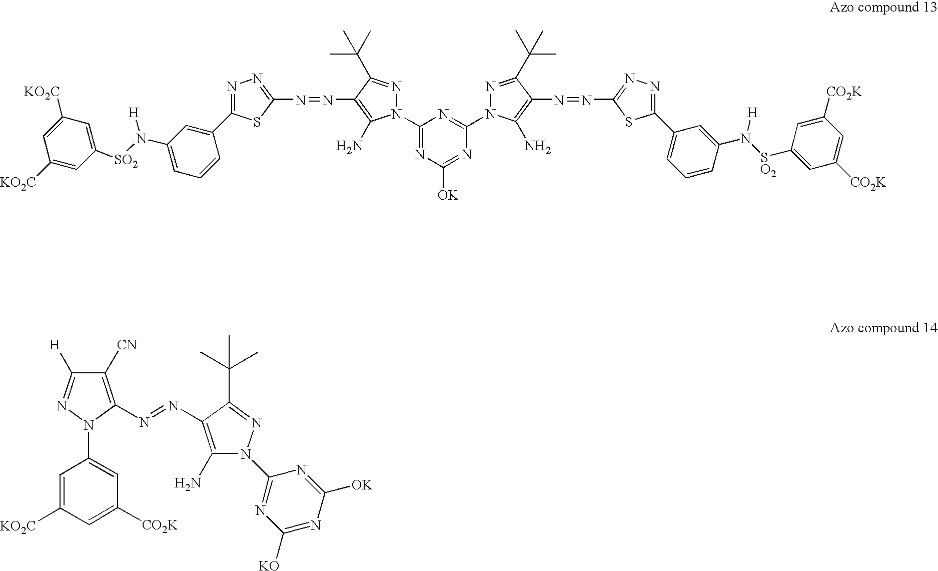

### Embodiment

Then, the invention is to be described more specifically by way of examples.

### Example 1

### Production Process for Exemplified Compound (D-9) of Formula (I)

### (I) Production Process for Intermediate Product A

An intermediate product A was synthesized in accordance with a method described in JP-A No. 2006-57076.

### (2) Production Process for Exemplified Compound (D-9)

500 mL of water was added to 20.0 g (127 mmol) of an intermediate product A, and 34.9 g (279 mmol) of pivaloylacetonitrile, they were stirred at a room temperature (internal temperature: 24° C., pH: 5.08), and 56 mL (56 mmol) of an aqueous 1 M solution of hydrochloric acid was added to a liquid reaction mixture (internal temperature: 25° C., pH upon charging: pH 2.81). After stirring at an internal temperature of 50° C. for 8 hr (internal temperature: 50° C., pH: 1.20), when 27 to 28 mL of an aqueous 8 M solution of potassium hydroxide was dropped slowly, the reaction solution formed a homogenous system (internal temperature: 50° C., pH: 8.7 to 8.9). Further, after stirring for 30 min at an internal temperature of 50° C., when 13.5 mL of an aqueous 12 M solution of hydrochloric acid was dropped slowly, crystals were precipitated (internal temperature: 50° C., pH: 6.5 to 6.7). The crystals were collected by filtration and rinsed with 400 mL of water and dried to obtain 40.2 g of Exemplified Compound (D-9) (separation yield: 85.1%, HPLC purity: 97.1%).

¹H-NMR (400 MHz, d6-DMSO) 5.32 ppm (s, 2H), 1.21 ppm (s, 18H)

### Comparative Example 1

A synthesis method for the 5-aminopyrazole (c1) from the hydrazine compound (b1) is described in JP-A 2006-057076. However, although a water solvent is used, since the reaction is conducted under an alkaline condition, the separation yield was as low as 15%.

### Example 2

Then, reaction was conducted while changing the addition amount of hydrochloric acid under various pH conditions.

In accordance with the following experimental examples, the residual rate of the intermediate product A and the yield of formation of the Exemplified Compound (D-9) were determined according to the area (%) at 254 nm by HPLC measurement.

### Experimental Example

Water was added by X (mL) to 1.0 g (6.28 mmol) of an intermediate product A and 1.75 g (14.0 mmol) of pivaloylacetonitrile, they were stirred at a room temperature, and Y (mL) of an aqueous 12M solution of hydrochloric acid was added to the mixed solution (internal temperature: 25° C., result of pH measurement described in Table 1). The reaction solution after 4, 8, 10 hr was sampled at an internal temperature of 50° C. and the residual rate of the intermediate product A and the yield of formation of Exemplified Compound (D-9) were determined according to the measurement HPLC.

TABLE 1

| | | | | | HPLC area (%) | | |
|---|---|---|---|---|---|---|---|
| | X | Y | pH upon | Reaction | Intermediate | | |
| | (mL) | (mL) | charging | time | product A | (D-9) | By-products |
| Comp. | 20 | 1.63 | 0.48 | 4 h | 16% | 66% | 18% |
| Example 1 | | | | 10 h | About equal with that for 4 h | | |
| Comp. | 20 | 0.70 | 1.45 | 4 h | 8% | 76% | 16% |
| Example 2 | | | | 10 h | About equal with that for 4 h | | |
| Invention 1 | 25 | 0.23 | 2.82 | 4 h | 18% | 75% | 7% |
| | | | | 10 h | 3% | 92% | 5% |
| Invention 2 | 25 | 0.12 | 3.04 | 4 h | 22% | 72% | 6% |
| | | | | 8 h | 3% | 96% | 1% |
| Comp. | 20 | 0.05 | 4.50 | 4 h | — | Trace amount | |
| Example 3 | | | | 8 h | 55% | 5% | 40% |

As shown in the table 1 described above it can be seen that, in a case where the pH upon charging the liquid mixture containing the hydrazine compound represented by Formula (II), the compound represented by Formula (III), the reaction solvent, and the acidifying agent is within a range from 2.5 to 3.0 (25° C.), while the reaction rate lowers somewhat the yield of formation of the Exemplified Compound (D-9) is improved. In comparative examples 1, 2, by-products are 10% or more and the yield of formation of (D-9) is not improved even by the reaction for 10 hr.

The 5-aminopyrazole derivative substituted by the water-soluble group represented by Formula (I) corresponds to (c1) in ka 16 on page 27 of JP-A No. 2006-57076, which is useful as an intermediate synthesis product for dyes, etc. represented as (Dye 62) of the patent publication due to (5) or the like described on page 28 thereof.

### Example 3

The synthesis scheme for the 5-aminopyrazole derivative substituted by the water-soluble group represented by Formula (4) (Exemplified Compound D-12) is shown below.

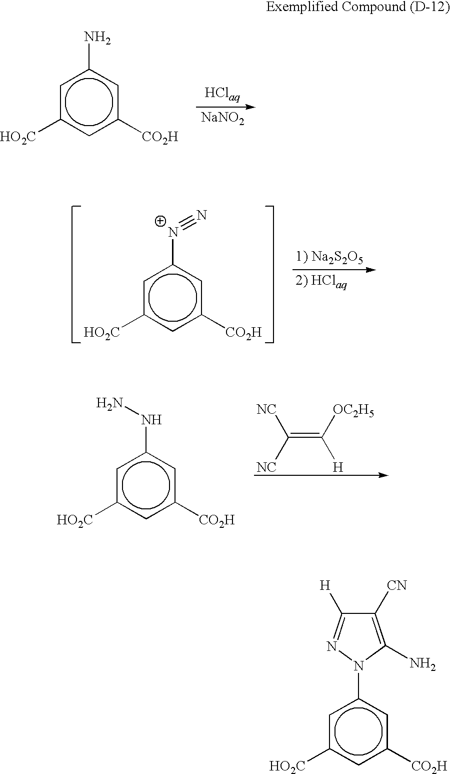

### Production Example 1

### Production Process for Exemplified Compound (D-12) of Formula (4)>

200 mL of water was added to 36.2 g (0.2 mol) of 5-aminoisophthalic acid and stirred at a internal temperature of 40° C., and 33.3 mL (0.4 mol) of an aqueous 12 M hydrochloric acid was added. They were completely solved by stirring at an internal temperature of 40° C. for 10 min and then cooled to an internal temperature of 0° C. (reaction solution A). Then, an aqueous solution formed by dissolving 13.8 g (0.2 mol) of sodium nitrite in 41.4 mL of water was dropped to the reaction solution A for 20 min (internal temperature was kept at 5° C. or lower). After stirring at an internal temperature of 2° C. for 1 hr, 1.2 g of sulfamic acid was divisionally added for 5 min (with bubbling) and further stirred at an internal temperature of 5° C. for 15 min to prepare an aqueous solution of a diazonium salt.

80 mL of water was added to 49.4 g (0.26 mol) of sodium disulfite Na₂S₂O₅ and completely dissolved by stirring at a room temperature and cooled to an internal temperature of 5° C. or lower. The aqueous solution of the diazonium salt was divisionally added to the mixed solution for 15 min while keeping the internal temperature at 15° C. or lower (the reaction vessel for the aqueous solution of the diazonium salt was cleaned thoroughly by using 25 mL of water). After stirring the reaction solution for 15 min at an internal temperature of 15° C. and for 60 min at the internal temperature of 50° C., 29.2 mL (0.35 mol) of an aqueous 12 M solution of hydrochloric acid was added and further stirred for 3 hours at an internal temperature of 85° C. The reaction solution was cooled to 40° C., and a nitrogen gas was caused to flow for 20 min to obtain an aqueous solution containing a hydrazine compound.

When 65 mL of an aqueous 50 mass % solution of sodium hydroxide was dropped to the aqueous solution containing the hydrazine compound, the reaction solution became homogeneous (internal temperature: 50° C., pH: 7.2). After adding 100 mL of methanol and 21.4 g (0.175 mol) of ethoxymethylene malononitrile (manufactured by Aldrich Co.) to the reaction solution and stirring for 1 hr at an internal temperature of 50° C., when a mixed solution of 18 mL of sulfuric acid and 18 mL of water was added, crystals were precipitated. After further stirring for 30 min at an internal temperature of 70° C., they were cooled to an internal temperature of 40° C., the crystals were collected by filtration, washed with 300 mL of water, further washed with a mixed solution of 100 mL of water and 100 mL of isopropyl alcohol, and dried to obtain 47.1 g of Exemplified Compound (D-12) (separation yield: 86.5%, HPLC purity: 97% or higher).

### Production Example 2

### Production Process for Exemplified Compound (D-12) of Formula (4)>

36.2 g (0.2 mol) of 5-aminoisophthalic acid, 181 mL of water, and 66.7 mL (0.8 mol) of aqueous 12 M hydrochloric acid were added at a room temperature and then cooled to an internal temperature of 1.5° C. (reaction solution A). Then, an aqueous solution formed by dissolving 13.8 g (0.2 mol) of sodium nitrite in 34.5 mL of water was dropped to the reaction solution A for 20 min (internal temperature was kept at 20° C. or lower). After stirring at an internal temperature of 2° C. for 1 hr, 1.2 g of sulfamic acid was divisionally added for 15 min (with bubbling) and further stirred at an internal temperature of 20° C. for 15 min to prepare an aqueous solution of a diazonium salt.

108 mL of water was added to 63.0 g (0.5 mol) of sodium disulfite and cooled to an internal temperature of 20° C. or lower. The aqueous solution of the diazonium salt was divisionally added to the mixed solution for 10 min while keeping the internal temperature at 25° C. or lower (the reaction vessel for the aqueous solution of the diazonium salt was cleaned thoroughly by using 12 mL of water). After stirring the reaction solution for 15 min at an internal temperature of 25° C. and for 60 min at an internal temperature of 50° C., 83.3 mL (1.0 mol) of an aqueous 12 M solution of hydrochloric acid was added and further stirred for 2 hours at an internal temperature of 70° C. The reaction solution was cooled to 40° C., to obtain an aqueous solution containing a hydrazine compound.

When 204 mL of an aqueous 8 M solution of sodium hydroxide was dropped to the aqueous solution containing the hydrazine compound, the reaction solution became homogeneous (internal temperature: 50° C., pH: 7.2). 24.4 g (0.2 mol) of ethoxymethylene malononitrile (manufactured by Aldrich Co.) was added to the reaction solution for 1.5 hr at an internal temperature of 60° C. (reaction solution B).

Then, the reaction solution B was dropped for 30 min to a mixed solution formed by stirring a mixed solution of 24 mL of sulfuric acid and 100 mL of water at an internal temperature of 60° C. (reaction vessel was washed thoroughly by using 20 mL of water three times). After stirring the reaction solution for 1 hr at an internal temperature of 70° C., it was cooled to 30° C. or lower, and crystals were collected by filtration, washed with 400 mL of water and further washed with 200 mL of isopropyl alcohol, and dried to obtain 46.6 g of an Exemplified Compound (D-12) (separation yield: 85.6%, HPLC purity: 98.0%).

### Example 4

A synthesis scheme for the 5-aminopyrazol derivative substituted by the water-soluble group represented by Formula (4) (Exemplified Compound D-34) in a case of using a reaction mixture of the compound shown by Formula (3) is to be shown below.

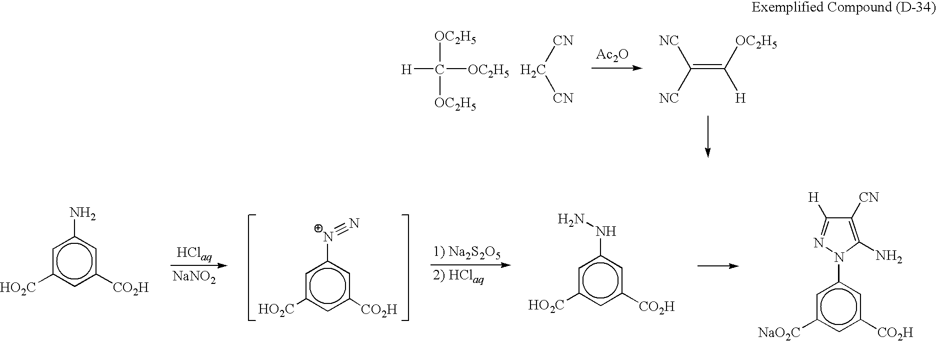

### Production Example 3

### Production Process for Exemplified Compound (D-34) of Formula (4)>

200 mL of water was added to 36.2 g (0.2 mol) of 5-aminoisophthalic acid and stirred at an internal temperature of 40° C., and 33.3 mL (0.4 mol) of an aqueous 12 M hydrochloric acid was added. They were completely dissolved by stirring for 10 min at an internal temperature of 40° C. and then cooled to an internal temperature of 0° C. (reaction solution A). Then, an aqueous solution formed by dissolving 13.8 g (0.2 mol) of sodium nitrite in 41.4 mL of water was dropped to the reaction solution A for 20 min (internal temperature was kept at 5° C. or lower). After stirring for 1 hr at an internal temperature of 2° C., 1.2 g of sulfamic acid was divisionally added for 5 min (with bubbling) and further stirred for 15 min at an internal temperature of 2° C. to prepare an aqueous solution of a diazonium salt.

80 mL of water was added to 49.4 g (0.26 mol) of sodium disulfite and completely dissolved by stirring at a room temperature and cooled to an internal temperature of 5° C. or lower. The aqueous solution of the diazonium salt was divisionally added to the mixed solution for 15 min while keeping the internal temperature at 15° C. or lower (the reaction vessel for the aqueous solution of the diazonium salt was cleaned thoroughly by using 25 mL of water). After stirring the reaction solution for 15 min at an internal temperature of 15° C. and for 60 min at an internal temperature of 50° C., 29.2 mL (0.35 mol) of an aqueous 12 M solution of hydrochloric acid was added and further stirred for 3 hours at an internal temperature of 85° C. The reaction solution was cooled to 40° C., and a nitrogen gas was caused to flow for 20 min to obtain an aqueous solution containing a hydrazine compound.

36.1 g (0.25 mol) of triethyl ortho-formate, 11.6 g (0.18 mol) of malononitrile, and 35.7 g (0.35 mol) of acetic acid anhydride were stirred under overheating at an internal temperature of 95° C. In this case, by-produced ethyl acetate was distilled off at a normal pressure. After stirring under overheating for 3 hrs, they were cooled to 30° C. to obtain a reaction mixture A.

When 64 to 66 mL of an aqueous 50 mass % solution sodium hydroxide was dropped to the aqueous solution containing the hydrazine compound, the reaction solution became homogeneous (internal temperature: 50° C., pH: 7.2-7.5). The reaction mixture A described above was added to the reaction solution. pH was 4.5 in this case. After stirring for 1 hr at an internal temperature of 50° C., precipitated crystals were collected by filtration, washed with 300 mL of water, further washed with mixed solution of 100 mL of water and 100 mL isopropyl alcohol, and dried to obtain 43.6 g of Exemplified Compound (D-34) (separation yield: 80.0%, HPLC purity: 97% or higher).

### Comparative Example 4

A production process for obtaining Exemplified Compound (D-12) of Formula (4) of the invention by isolating, from 5-aminoisophthalic acid, a corresponding hydrazine compound is described in JP-A No. 2006-057076. According to the production process, it is described that the corresponding hydrazine compound was obtained from the 5-aminoisophthalic acid at a yield of 68.8%, and Exemplified Compound (D-12) was obtained from the hydrazine compound at 86.3%.

As described above, in the method of isolating the hydrazine compound described in JP-A No. 2006-057076, the yield in two steps of Exemplified compound of (D-12) of Formula (4) of the invention from 5-aminoisophthalic acid is 59.4%, and it can be seen that the yield is lower compared with the invention. Further, as shown in Production Example 2, since Exemplified Compound (D-12) of Formula (4) can be produced from 5-aminoisophthalic acid always by using the solvent consisting only of water, this is a production process giving less burden on the environment.

### Example 5

### Change of HPLC Area (%) of the Reaction Solution of 5-aminopyrazole Derivative Depending on pH

After adding an aqueous 50 mass % solution of sodium hydroxide to the aqueous solution containing the hydrazine compound obtained in Example 3 and adjusting to the pH shown in the following Table 2, ethoxymethylene malononitrile was added and HPCL areas (%) after stirring for 1 hr at an internal temperature of 50° C. are shown in the following Table 2.

Further, after adding an aqueous 50 mass % solution of sodium hydroxide to the aqueous solution containing the hydrazine compound obtained in Example 4 and adjusting to the pH shown in the following Table 2, the reaction mixture A of the compound shown by Formula (3) was added and HPCL areas (%) after stirring at an internal temperature of 50° C. for 1 hr are shown in the following Table 2.

TABLE 2

| | | Reaction solution pH | HPLC area (%) |
|---|---|---|---|
| Example 3-1 | Invention | 7.2 | 91% |
| Example 3-2 | Invention | 6.0 | 87% |
| Example 3-3 | Invention | 4.2 | 75% |
| Example 4-1 | Invention | 7.2 | 88% |
| Example 4-2 | Invention | 6.0 | 85% |
| Example 4-3 | Invention | 4.2 | 84% |

Since ethoxymethylene malononitrile is gradually decomposed in an alkaline aqueous solution, pH is desirably at 9 or lower. Further, from the result of Table 2 it can be seen that 5-aminopyrazole derivatives can be obtained at a high yield of formation when the reaction of the hydrazine compound represented by Formula (2) and the compound represented by Formula (3) is conducted within a pH range of the reaction solution of from 5 to 9 in the step of (C). In a case of using the reaction mixture A shown by Formula (3), a satisfactory 5-aminopyrazole derivative can be obtained at a high yield of formation within a pH range of from 4.2 to 7.2.

### Example 6

A synthesis example of azo dyes (azo compounds 1 to 3) using the compound D-34 of Formula (4) produced by the production method of the invention is to be shown below.

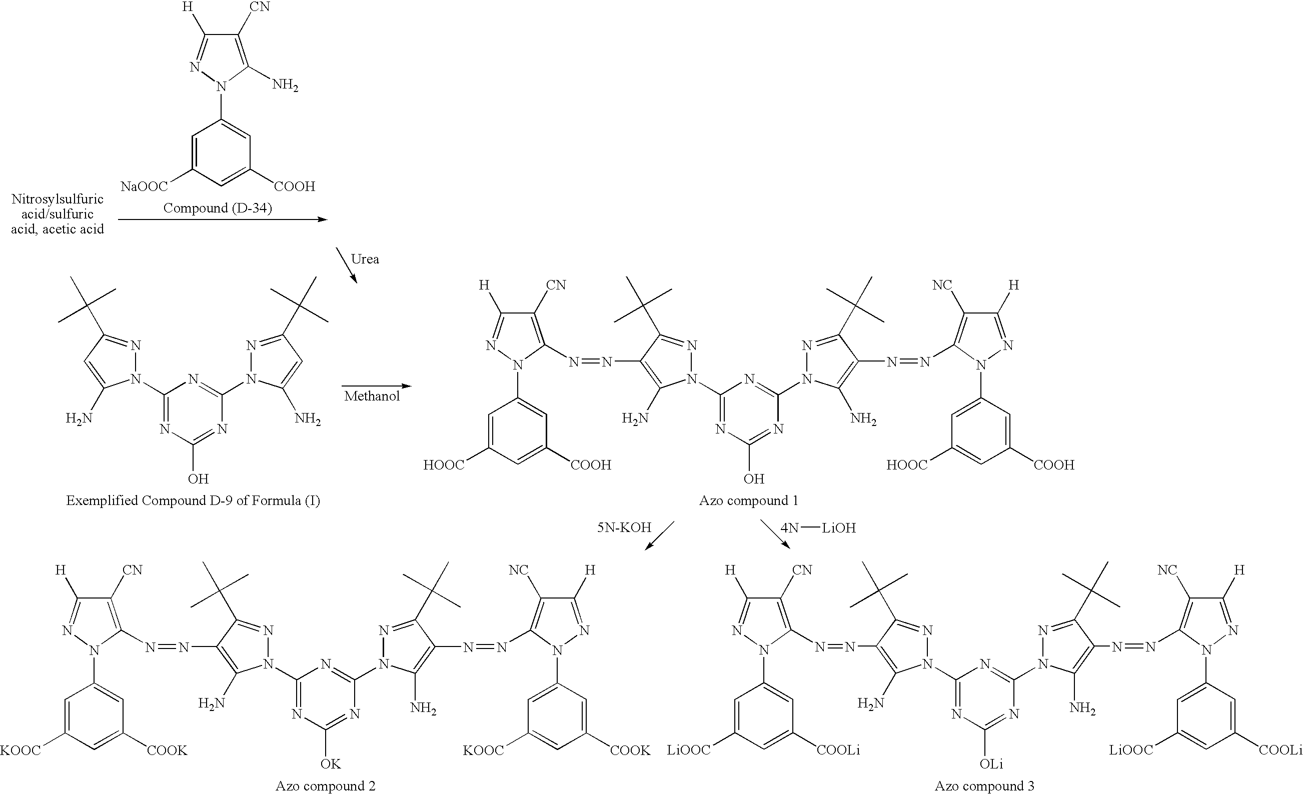

After stirring 9.2 g of 95% sulfuric acid at an internal temperature of 10° C., 23.6 g of acetic acid was added and the internal temperature was kept at 10° C. or lower, to which 4.9 mL of 40 mass % nitrosylsulfuric acid (manufactured by Aldrich Co.) was dropped. Further, 8.0 g of compound D-34 was divisionally added and stirred for 1.5 hr at an internal temperature of 0 to 5° C. 0.6 g of urea was added to the reaction solution and, further, stirring was continued for 0.5 hr (diazonium compound preparing solution). A diazonium compound preparing solution was dropped at an internal temperature of 20° C. or lower to 68 mL of a methanol solution containing 5.4 g of the exemplified compound (D-9) of Formula (I) separately produced by the production method of the invention with addition of 5.7 mL of methanol solution containing 1.6 g of potassium hydroxide (internal temperature: 20° C.). After dropping, the internal temperature was elevated to 20° C. and stirring for 1 hr, they were cooled to 10° C. After adding 26.3 mL of separately prepared methanol solution containing 7.4 g of potassium hydroxide at 20° C. or lower, the internal temperature was elevated to 50° C. and they were stirred for 1 hr while keeping the temperature. pH was 0.6 in this case. Then, they were cooled to 30° C. or lower for 1 hr and crystallized yellow crystals (azo compound 1) was filtered under suction by using filter paper 70 mm (type 2) manufactured by ADVANTEC Co. The time required for filtration was 30 sec and yield of formation of the azo compound 1 was 93.2% (HPCL area %).

The obtained crystals were suspended in 130 mL of ion exchange water and, after elevating the temperature to 70° C., stirred for 1 hr and filtered under suction to obtain 10.8 g (yield: 94.0%). 10.0 g of the yellow crystals was suspended and dispersed in 47 g of ion exchange water, and 11 mL of an aqueous 5 mol/L of potassium hydroxide solution was dropped at a room temperature to adjust pH to 7.5. After elevating the temperature of the solution to an internal temperature of 50° C., 223 mL of isopropanol was added and cooled till the internal temperature was lowered to 30° C. Crystallized yellow crystals were filtered under suction and rinsing was conducted with 223 mL of isopropanol. After drying at 75° C., 11.6 g of azo compound 2 was obtained (yield 96.4%) λmax: 455.3 nm (H₂O/dimethylformamide=2/98), ε: 37327.

In the same manner, azo compound 1 was suspended in 130 mL of ion exchanged water and, after elevating the temperature to 70° C., it was stirred for 1 hr and filtered under suction to obtain 10.2 g (yield: 88.8%). 10.0 g of yellow crystals was suspended and dispersed in 47 g of ion exchange water, and 8.8 mL of an aqueous 4 mol/L lithium hydroxide solution was dropped at a room temperature to adjust pH to 7.5. After elevating the temperature of the solution to an internal temperature of 50° C., 223 mL of isopropanol was added and cooled till the internal temperature was lowered to 30° C. Crystallized yellow crystals were filtered under suction and rinsing was conducted with 223 mL of isopropanol. After drying 75° C., 10.0 g of azo compound 3 was obtained (yield 97.0%) λmax: 454.8 nm (H₂O/dimethylformamide=2/98), ε: 37553.

### Example 7

Azo dyes (azo compounds 4 to 14) were produced by the same method as described in Example 6. Specifically, corresponding azo dyes were produced by using the methods of synthesizing azo dyes described, for example, in the pamphlet of WO No. 2005/7553, JP-A Nos. 2007-063520 and 2007-217681 each alone or, optionally, as a combination of them. The azo compounds 4 to 11 and 14 were produced by fixing the diazo component to the compound D-34 represented by Formula (4) produced by the production method of the invention, and optionally changing the coupler component. Further, the azo compounds 12 and 13 were produced by fixing the coupler component to the exemplified compound (D-9) of the Formula (I) and changing the diazo component. The following Table 3 shows the result of measurement for λmax and max of the obtained azo compound in water.

TABLE 3

| Azo Compound | λmax | εmax |
|---|---|---|
| 4 | 448 | 17900 |
| 5 | 459 | 22500 |
| 6 | 458 | 22600 |
| 7 | 444 | 19000 |
| 8 | 503 | 27700 |
| 9 | 436 | 12600 |
| 10 | 439 | 16800 |
| 11 | 437 | 18900 |
| 12 | 452 | 58800 |
| 13 | 457 | 61000 |
| 14 | 442 | 19400 |

### INDUSTRIAL APPLICABILITY

According to the production process of the invention, the 5-aminopyrazole derivative (coupler component) substituted with the water-soluble group represented by Formula (1) is obtained at a high yield and a high purity in an aqueous system which is inexpensive and gentle to the environment.

Further, according to the production process of the invention, the 5-aminopyrazole derivative (diazo component) substituted with the water-soluble group represented by Formula (4) can be obtained simply and conveniently at a high yield and a high purity. Further, since all of the steps (A) to (C) can also be conducted with a solvent consisting only of water, this is a production process giving less burden on the environment.

Further, by using the 5-amnopyrazole derivative according to production process of the invention as the intermediate product for an azo dye, the hue and the color can be improved remarkably.

The entire disclosure of each and every foreign patent application from which the benefit of foreign priority has been claimed in the present application is incorporated herein by reference, as if fully set forth.

1. A process for producing a 5-aminopyrazole derivative represented by Formula (I), comprising:
a step of reacting a hydrazine compound represented by Formula (II) with a compound represented by Formula (III) under a condition satisfying Reaction Condition 1 and Reaction Condition 2, wherein
Reaction Condition 1 is that the reaction is conducted in a reaction solvent containing water, and
Reaction Condition 2 is that pH of a liquid mixture containing the hydrazine compound represented by Formula (II), the compound represented by Formula (III), the reaction solvent, and an acidifying agent upon charging is within a range of from 2.0 to 4.0 at 25° C.: 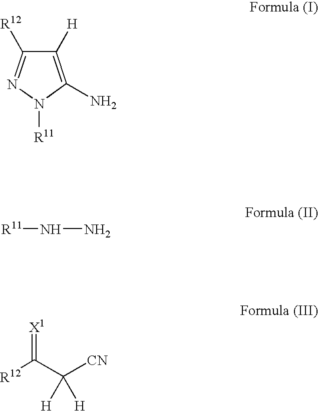
wherein
R¹¹ represents an aromatic group or a heterocyclic group, each of which is substituted with a hydroxyl group, a carboxyl group, or a sulfo group,
R¹² represents an aliphatic group, aromatic group, or heterocyclic group each of which is substituted or unsubstituted, and
X¹ represents an oxygen atom or NH.

2. The production process according to claim 1, wherein
R¹¹ in Formula (I) and Formula (II) is a heterocyclic group substituted with a hydroxyl group.

3. The production process according to claim 1, wherein
the acidifying agent is sulfuric acid or hydrochloric acid.

4. The production process according to claim 1, wherein
the reaction is conducted at a temperature of from 30 to 70° C.

5. The production process according to claim 1, wherein
the reaction solvent containing water is a solvent consisting only of water.

6. The production process according to claim 1, wherein
pH of the liquid mixture upon charging is within a range of from 2.5 to 3.5 at 25° C.

7. A process for producing a 5-aminopyrazole derivative represented by Formula (4) comprising:
(A) a step of diazotizing an amine compound represented by Formula (1) to prepare a diazonium salt;
(B) a step of reducing the diazonium salt to prepare a hydrazine compound represented by Formula (2); and
(C) a step of reacting the hydrazine compound with a compound represented by Formula (3) to prepare a 5-aminopyrazole derivative represented by Formula (4),
wherein the steps (A) to (C) are conducted continuously: 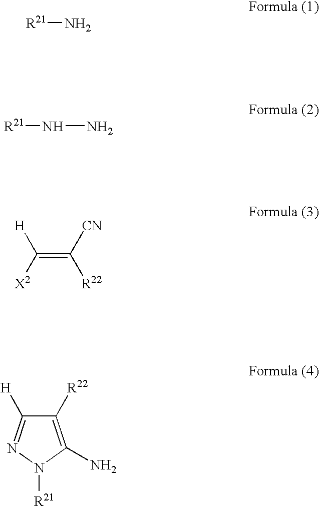
wherein
R²¹ represents an aromatic group or a heterocyclic group, each of which is substituted with a hydroxyl group, carboxyl group or sulfo group,
R²² represents an electron-withdrawing group, and
X² represents a halogen atom, hydroxyl group, amino group, or alkoxy group.

8. The production process according to claim 7, wherein
the steps (A) and (B) are conducted with a solvent consisting only of water.

9. The production process according to claim 7, wherein
all the steps (A), (B) and (C) are conducted with a solvent consisting only of water.

10. The production process according to claim 7 wherein
the reducing agent for reducing the diazonium salt in the step (B) is sodium sulfite or sodium disulfate.

11. The production process according to claim 7,
the reducing agent for reducing the diazonium salt in the step (B) is added in an amount of from 1.2 to 3.0 mol based on 1 mol of the amine compound represented by Formula (1).

12. The production process according to claim 7, wherein
the step (C) is conducted in a reaction solution, pH of which is within a range of from 5.0 to 9.0.

13. The production process according to claim 7, further comprising after the step(C):
a step of crystallizing the 5-aminopyrazole derivative represented by Formula (4) prepared in the step (C) at pH of the reaction solution within a range of from 0.5 to 2.0, and
a step of collecting the crystallized 5-aminopyrazole derivative by filtration.

14. A production process according to claim 7, wherein
a reaction mixture obtained by reacting a compound represented by Formula (6) with a compound represented by a Formula (7) is used instead of the compound represented by Formula (3) in the step (C): 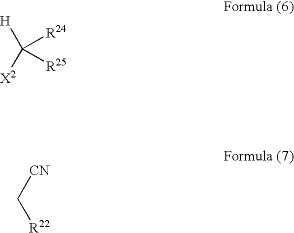
wherein
R²² represents an electron-withdrawing group,
X² represents a halogen atom, a hydroxyl group, amino group, or alkoxy group, and
R²⁴ and R²⁵ each represents independently a group leaving easily by the reaction of the compounds.

15. The production process according to clam 14, wherein
the step (C) is conducted in a reaction solution, pH of which is within a range of from 3.0 to 9.

16. A production process according to claim 14, further comprising after the step (C):
a step of crystallizing the 5-aminopyrazole derivative represented by Formula (4) in the step (C) at pH of the reaction solution within a range of from 3.0 to 5.0 and
a step of collecting the crystallized 5-aminopyrazole derivative by filtration.

17. An azo dye produced from the 5-aminopyrazole derivative produced by the production process according to claim 1.

18. A process for producing an azo dye, comprising:
a step of producing a 5-aminopyrazole derivative represented by Formula (I), comprising:
a step of reacting a hydrazine compound represented by Formula (II) with a compound represented by Formula (III) under a condition satisfying Reaction Condition 1 and Reaction Condition 2, wherein
Reaction Condition 1 is that the reaction is conducted in a reaction solvent containing water, and
Reaction Condition 2 is that pH of a liquid mixture containing the hydrazine compound represented by Formula (II), the compound represented by Formula (III), the reaction solvent, and an acidifying agent upon charging is within a range of from 2.0 to 4.0 at 25° C.: 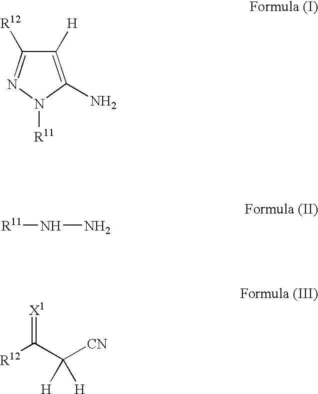
wherein
R¹¹ represents an aromatic group or a heterocyclic group, each of which is substituted with a hydroxyl group, a carboxyl group, or a sulfo group,
R¹² represents an aliphatic group, aromatic group, or heterocyclic group each of which is substituted or unsubstituted, and
X¹ represents an oxygen atom or NH;
a step of producing a 5 aminopyrazole derivative represented by Formula (4) comprising:
(A) a step of diazotizing an amine compound represented by Formula (1) to prepare a diazonium salt,
(B) a step of reducing the diazonium salt to prepare a hydrazine compound represented by Formula (2), and
(C) a step of reacting the hydrazine compound with a compound represented by Formula (3) to prepare a 5-aminopyrazole derivative represented by Formula (4),
wherein the steps (A) to (C) are conducted continuously: 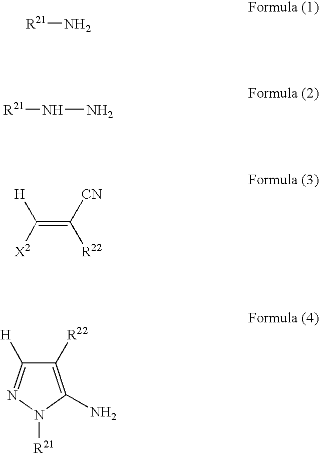
wherein
R²¹ represents an aromatic group or a heterocyclic group, each of which is substituted with a hydroxyl group, carboxyl group or sulfo group,
R²² represents an electron-withdrawing group, and
X² represents a halogen atom, hydroxyl group, amino group, or alkoxy group; and
a step of reacting the 5-aminopyrazole derivative represented by Formula (I) with the 5-aminopyrazole derivative represented by Formula (4).

19. An azo dye produced from the 5-aminopyrazole derivative produced by the production process according to claim 7.
